(19) United States
(12) Patent Application Publication
(10) Pub. No.: US 2012/0128730 A1
(43) Pub. Date: May 24, 2012

(54) COMPOSITIONS AND METHODS FOR ONCE-DAILY TREATMENT OF OBSESSIVE COMPULSIVE DISORDER WITH ONDANSETRON

(76) Inventors: NIPUN DAVAR, Pleasanton, CA (US); Pauly Kavalakatt, Fremont, CA (US); Sangita Ghosh, Foster City, CA (US)

(21) Appl. No.: 12/975,215

(22) Filed: Dec. 21, 2010

Related U.S. Application Data
(60) Provisional application No. 61/416,685, filed on Nov. 23, 2010.

(51) Int. Cl. A61K 31/4178; A61P 25/00; A61K 9/00

(52) U.S. Cl. 424/400; 514/397

(57) ABSTRACT

Compositions for once-daily administration of ondansetron are described. Compositions include a core, a semi-permeable membrane disposed generally around the core, an orifice in the semi-permeable membrane in fluid communication with the core, and a coating having a first therapeutically effective dose of ondansetron or a pharmaceutically acceptable salt disposed generally around the semi-permeable membrane. The core includes first, second, and third layers. The first layer is in fluid communication with the orifice. The second layer includes a second therapeutically effective dose of ondansetron or a pharmaceutically acceptable salt and is located adjacent to the first layer. The third layer is located adjacent the second layer. Methods for treating obsessive compulsive disorder by administering these compositions are also described.

### CROSS-REFERENCES TO RELATED APPLICATIONS

This application claims priority to U.S. Application Ser. No. 61/416,685, filed Nov. 23, 2010, entitled “Compositions and Methods for Once-Daily Treatment of Obsessive Compulsive Disorder with Ondansetron.” The subject matter of this application is also related to U.S. application Ser. No. 12/782,571, filed May 18, 2010, which claims priority to U.S. Provisional Application Ser. No. 61/179,439, filed May 19, 2009, entitled “Method of Treatment of Obsessive Compulsive Disorder with Ondansetron.” All of above-mentioned applications are hereby expressly incorporated by reference in their entirety for all purposes.

### BACKGROUND OF THE INVENTION

Obsessive compulsive disorder (“OCD”) is a mental condition that is most commonly characterized by intrusive, repetitive unwanted thoughts (obsessions) resulting in compulsive behaviors and mental acts that an individual feels driven to perform (compulsion). Current epidemiological data indicates that OCD is the fourth most common mental disorder in the United States. Some studies suggest the prevalence of OCD is between one and three percent, although the prevalence of clinically recognized OCD is much lower, suggesting that many individuals with the disorder may not be diagnosed. Patients with OCD are often diagnosed by a psychologist, psychiatrist, or psychoanalyst according to the DSM-IV-TR (2000) diagnostic criteria that include six characteristics of obsessions and compulsions:

Obsessions
Recurrent and persistent thoughts, impulses, or images that are experienced as intrusive and that cause marked anxiety or distress.
The thoughts, impulses, or images are not simply excessive worries about real-life problems.
The person attempts to ignore or suppress such thoughts, impulses, or images, or to neutralize them with some other thought or action.
The person recognizes that the obsessional thoughts, impulses, or images are a product of his or her own mind, and are not based in reality.

Compulsions
Repetitive behaviors or mental acts that the person feels driven to perform in response to an obsession, or according to rules that must be applied rigidly.
The behaviors or mental acts are aimed at preventing or reducing distress or preventing some dreaded event or situation; however, these behaviors or mental acts are not actually connected to the issue, or they are excessive.

Individuals with OCD typically perform tasks (or compulsion) to seek relief from obsession-related anxiety. Repetitive behaviors such as hand washing, counting, checking, or cleaning are often performed with the hope of preventing obsessive thoughts or making them go away. Performing these “rituals,” however, only provides temporary relief. People with OCD may also be diagnosed with a spectrum of other mental disorders, such as generalized anxiety disorder, anorexia nervosa, panic attack (http://en.wikipedia.org/wiki/Social_anxiety_disorder), or schizophrenia.

Studies suggest that OCD may be related to abnormal levels of a neurotransmitter called serotonin. The first-line treatment of OCD consists of behavioral therapy, cognitive therapy, and medications. Medications for treatment include selective serotonin reuptake inhibitors (SRIs or SSRIs) such as paroxetine (Seroxat™, Paxil®, Xetanor™, ParoMerck™, Rexetin™), sertraline (Zoloft®, Stimuioton™), fluoxetine (Prozac®, Bioxetin™), escitalopram (Lexapro®), and fluvoxamine (Luvox®) as well as the tricyclic antidepressants, in particular clomipramine (Anafranil®). Benzodiazepines are also used in treatment. As much as 40 to 60% of the patients, however, fail to adequately respond to the SRI therapy and an even greater proportion of patients fail to experience complete remission of their symptoms.

As a second-line treatment for those patients (50-60%) who do not respond to first-line treatment (SSRIs), atypical antipsychotics (or neuropleptics) can be used as augmentation in treatment-resistant OCD. These atypical antipsychotics block central dopamine receptors and may be helpful in blocking mid-brain dopamine receptors. Low doses of atypical antipsychotics, such as olanzapine (Zyprexa®), quetiapine (Seroquel®), ziprasidone, and risperidone (Risperdal®), have been found useful as adjuvant therapy with SSRIs in the treatment of OCD. Other alternative agents, including inositol, vitamin supplement, opioids, hallucinogen LSD, and nicotine, have been attempted as a drug treatment for OCD. The efficacy of the adjunctive antipsychotics with SSRIs may be due to direct D₂ blockade separate or together with antagonism of 5-HT₂ receptors. These patients may also suffer from additional abnormalities in dopaminergic function that require augmentation with these dopamine-blocking agents. Still, only a third to half of patients with SRI-resistant (treatment-resistant) OCD had a beneficial response to this second-line treatment.

Obsessive-compulsive disorder treatment can, however, be difficult. In fact, 30% of patients do not respond to adjuvant therapy (neuroleptics plus SSRIs) and are classified as treatment-resistant patients. For these patients, current therapies do not offer a cure.

Alternative therapies for treatment of OCD and treatment-resistant OCD are needed.

### SUMMARY OF THE INVENTION

The present invention provides methods and compositions for treating OCD, including treatment-resistant OCD, with ondansetron formulated in a multi-layer tablet for once-daily administration. Ondansetron could be used either in its free base forms or suitable salt form. The most commonly used salt form of ondansetron is ondansetron hydrochloride.

In one embodiment of the invention, the invention provides pharmaceutical compositions having a core, a semi-permeable membrane disposed generally around the core, an orifice in the semi-permeable membrane in fluid communication with the core, and a coating having a first therapeutically effective dose of ondansetron or a pharmaceutically acceptable salt disposed generally around the semi-permeable membrane. The core includes first, second, and third layers. The first layer is in fluid communication with the orifice. The second layer includes a second therapeutically effective dose of ondansetron or a pharmaceutically acceptable salt and is located adjacent to the first layer. The third layer is located adjacent the second layer. The first layer may have a higher viscosity than the second layer.

In another embodiment, the invention provides a method of treating obsessive compulsive disorder in a patient. Pharmaceutical compositions or dosage forms are provided that have a core, a semi-permeable membrane disposed generally around the core, an orifice in the semi-permeable membrane in fluid communication with the core, and a coating having a first therapeutically effective dose of ondansetron or a pharmaceutically acceptable salt disposed generally around the semipermeable membrane. The core includes first, second, and third layers. The first layer is in fluid communication with the orifice. The second layer includes a second therapeutically effective dose of ondansetron or a pharmaceutically acceptable salt and is located adjacent to the first layer. The third layer is located adjacent the second layer. The first layer may have a higher viscosity than the second layer. The pharmaceutical composition or dosage form is then administered to the patient suffering from OCD.

In another embodiment, the invention provides pharmaceutical compositions having a core, a semi-permeable membrane disposed generally around the core, an orifice in the semi-permeable membrane in fluid communication with the core, and a coating having a first therapeutically effective dose of ondansetron or a pharmaceutically acceptable salt disposed generally around the semi-permeable membrane. The core includes first, second, and third layers. The first layer is in fluid communication with the orifice. The second layer includes a second therapeutically effective dose of ondansetron or a pharmaceutically acceptable salt and is located adjacent to the first layer. The third layer is located adjacent the second layer. Each of the first and second layers include polyethylene oxide having an average molecular weight and the average molecular weight of the polyethylene oxide in the first layer may be higher than the average molecular weight of the polyethylene oxide in the second layer.

In another embodiment, the invention provides a method of treating obsessive compulsive disorder in a patient. Pharmaceutical compositions or dosage forms are provided that have a core, a semi-permeable membrane disposed generally around the core, an orifice in the semi-permeable membrane in fluid communication with the core, and a coating having a first therapeutically effective dose of ondansetron or a pharmaceutically acceptable salt disposed generally around the semi-permeable membrane. The core includes first, second, and third layers. The first layer is in fluid communication with the orifice. The second layer includes a second therapeutically effective dose of ondansetron or a pharmaceutically acceptable salt and is located adjacent to the first layer. The third layer is located adjacent the second layer. Each of the first and second layers include polyethylene oxide having an average molecular weight and the average molecular weight of the polyethylene oxide in the first layer may be higher than the average molecular weight of the polyethylene oxide in the second layer. The pharmaceutical composition or dosage form is then administered to the patient suffering from OCD.

In another embodiment, the invention provides a method of treating obsessive compulsive disorder in a patient. Pharmaceutical compositions or dosage forms are provided that have a core, a semi-permeable membrane disposed generally around the core, an orifice in the semi-permeable membrane in fluid communication with the core, and a coating comprising a first therapeutically effective dose of ondansetron or a pharmaceutically acceptable salt disposed generally around the semi-permeable membrane. The core includes first, second, and third layers, wherein the first layer is in fluid communication with the orifice, wherein the second layer comprises a second therapeutically effective dose of ondansetron or a pharmaceutically acceptable salt and is located adjacent to the first layer, and wherein the third layer is located adjacent the second layer.

In another embodiment, the invention provides a method of treating obsessive compulsive disorder in a patient. Pharmaceutical compositions or dosage forms are provided that include a first compartment and a second compartment. Each of the first and second compartments includes a therapeutically effective dose of ondansetron. The pharmaceutical composition or dosage form is then administered to the patient suffering from OCD. The first and second layers initiate release at different times and result in a first dose-normalized Cₘₐₓ, a first Tₘₐₓ, a second dose-normalized Cₘₐₓ, and a second Tₘₐₓ. The first and second dose-normalized Cₘₐₓ's may each be between about 0.1 ng/ml and about 6 ng/ml. The first Tₘₐₓ may be between about 0.1 hours and about 6 hours of administration and the second Tₘₐₓ may be between about 6 hours and about 20 hours of administration.

The present invention also includes methods for treating a subject suffering from OCD by administering to the subject a once-daily dosage form, e.g., a capsule-shaped tablet, containing a total dose of ondansetron or a pharmaceutically acceptable salt that releases the total dose of ondansetron in a bimodal distribution that results in a dose-normalized plasma ondansetron concentration of about 0.2 rig/ml to about 3 ng/ml within about 0.1 hours to about 5 hours and about 0.2 ng/ml to about 3 ng/ml within about 6 hours and 20 hours. Additionally, the two maxima of the ondansetron plasma concentrations may be separated by about 1 hour to about 12 hours, alternatively by about 2 hours to about 12 hours, alternatively by about 2 hours to about 10 hours, alternatively by about 2 hours to about 8 hours, alternatively by about 2 hours to about 6 hours. This bimodal profile provides therapeutic delivery above the minimum effective concentration while keeping the plasma levels below the maximum tolerated concentration and low enough such that side effects will be reduced. This delivery profile provides 24 hours of efficacy without initially undesirable high plasma levels.

In another embodiment, the invention provides a pharmaceutical composition having a core, a semi-permeable membrane disposed generally around or surrounding the core, an orifice in the semi-permeable membrane in fluid communication with the core, and a coating comprising a first therapeutically effective dose of ondansetron or a pharmaceutically acceptable salt disposed generally around or surrounding the semi-permeable membrane. The core may include first, second, and third layers. The first layer is in fluid communication with the orifice. The second layer has a second therapeutically effective dose of ondansetron or a pharmaceutically acceptable salt and is located adjacent to the first layer. The third layer is located adjacent the second layer.

For any of the dosage forms and pharmaceutical compositions of this invention, the coating may contain ondansetron in a dose of about 0.125 mg to about 1.0 mg, alternatively about 0.125 mg to about 0.75 mg, alternatively about 0.4 mg to about 0.6 mg. The coating may be generally disposed around or surround (at least partially or fully) a core of the tablet. The second layer may contain ondansetron in a dose of about 0.125 mg to about 1.0 mg, alternatively about 0.125 mg to about 0.75 mg, alternatively about 0.4 mg to about 0.6 mg.

For any of the dosage forms and pharmaceutical compositions of this invention, the coating, which contains a therapeutically effective amount of ondansetron and is generally, disposed around or surrounds (at least partially or full) the trilayer tablet core, may be formulated for release from the dosage form or composition within about 2 hours, alternatively within about 1.5 hours, alternatively within about 1 hour, alternatively within about 0.5 hours, alternatively within about 20 minutes, alternatively within about 15 minutes, alternatively within about 10 minutes of administration of the pharmaceutical composition or dosage form. The second layer, which contains a therapeutically effective amount of ondansetron and is part of a core of a tablet (e.g., one layer of a multi-layer tablet), may be formulated for release from the dosage form or composition in not less than about 8 hours, alternatively not less than about 9 hours, alternatively not less than about 10 hours, alternatively not less than about 11 hours, alternatively not less than about 12 hours, alternatively not less than about 13 hours, alternatively not less than about 14 hours, alternatively not less than about 15 hours, alternatively not less than about 16 hours after administration of the pharmaceutical composition or dosage form. Furthermore, there may be no substantial accumulation of ondansetron in the patient after 24-hours following administration.

For any of the dosage forms and pharmaceutical compositions of this invention, the first layer of the trilayer core may have a higher viscosity than the second layer. The first layer may have a viscosity between about 50 and about 20,000 cps, alternatively between about 50 and about 15,000 cps, alternatively between about 50 and about 10,000 cps, alternatively between about 50 and about 5,000 cps, alternatively between about 50 and about 2,500 cps, alternatively between about 50 and about 1,500 cps, alternatively between about 600 and about 1200 cps, alternatively between about 500 and about 1000 cps. The second layer of the trilayer core may have a viscosity between about 5 and about 10,000 cps, alternatively between about 5 and about 7,500 cps, alternatively between about 5 and about 5,000 cps, alternatively between about 5 and about 2,500 cps, alternatively between about 5 and about 1,000 cps, alternatively between about 5 and about 500 cps, alternatively between about 5 and about 100 cps, alternatively between about 30 and about 50 cps, alternatively between about 5 and about 50 cps.

For any of the dosage forms and pharmaceutical compositions of this invention, each of the first and second layers of the trilayer core may include polyethylene oxide having an average molecular weight and the average molecular weight of the polyethylene oxide in the first layer may be higher than the average molecular weight of the polyethylene oxide in the second layer. The average molecular weight of the polyethylene oxide in the first layer may be about 200K to about 1000K, alternatively about 200K to about 800K, alternatively about 200K, alternatively about 300K, alternatively about 400K, alternatively about 500K, alternatively about 600K, alternatively about 700K, alternatively about 800K, alternatively about 900K, alternatively about 1000K. The average molecular weight of the polyethylene oxide in the second layer may be about 100K to about 600K, alternatively about 100K to about 500K, alternatively about 100K to about 400K, alternatively about 100K to about 300K, alternatively about 100K, alternatively about 200K, alternatively about 300K, alternatively about 400K, alternatively about 500K, alternatively about 600K.

For any of the dosage forms and pharmaceutical compositions of this invention, the weight of the third layer may be ⅓ or more of the total weight of the trilayer core, alternatively ½ or more of the total weight of the trilayer core. Similarly, the weight of the first or second layer may be ⅓ or more of the total weight of the trilayer core, alternatively ½ or more of the total weight of the trilayer core. Furthermore, each of the first, second, and third layers may each comprise the same excipient, e.g., polyethylene oxide.

For any of the compositions or methods described herein, the dosage forms or pharmaceutical compositions of this invention may result in first and second Cₘₐₓ's, Tₘₐₓ's, Cₘᵢₙ's, and Tₘᵢₙ's that correspond to the release of the ondansetron in the first and second layers or compartments. The first and second dose-normalized Cₘₐₓ's may each independently be between about 0.1 ng/ml and about 8 ng/ml, alternatively between about 0.5 ng/ml and about 6 ng/ml, alternatively between about 1.0 ng/ml and about 5 ng/ml, alternatively between about 1.5 ng/ml and about 4.5 ng/ml. The first Tₘₐₓ, which corresponds to the release of the first therapeutically effective amount of ondansetron, may occur between about 0.1 hours and about 6 hours, alternatively between about 0.1 hours and 5 hours, alternatively between about 0.1 hours and 4 hours, alternatively between about 0.1 hours and 3 hours, alternatively between about 0.1 hours and 2 hours, alternatively between about 0.1 hours and 1 hour, alternatively between about 0.1 hours and 0.5 hours after administration of the dosage form or pharmaceutical composition. The second Tₘₐₓ, which corresponds to the release of the second therapeutically effective amount of ondansetron, may occur between about 5 hours and about 21 hours, alternatively between about 6 hours and 20 hours, alternatively between about 6 hours and 18 hours, alternatively between about 6 hours and 16 hours, alternatively between about 6 hours and 14 hours, alternatively between about 6 hours and 12 hours, alternatively between about 6 hours and 10 hours after administration of the dosage form or pharmaceutical composition. The first and second dose-normalized Cₘᵢₙ's may each independently be between about 0.1 ng/ml and about 3 ng/ml, alternatively between about 0.1 ng/ml and about 2 ng/ml, alternatively between about 0.1 ng/ml and about 1.0 ng/ml, alternatively between about 0.1 ng/ml and about 1.0 ng/ml, alternatively between about 0.1 ng/ml and about 0.8 ng/ml, alternatively between about 0.2 ng/ml and about 1.0 ng/ml, alternatively between about 0.3 ng/ml and about 1.0 ng/ml, alternatively between about 0.4 ng/ml and about 1.0 alternatively between about 0.5 ng/ml and about 1.0 ng/ml, alternatively between about 0.6 ng/ml and about 1.0 ng/ml, alternatively between about 0.7 ng/ml and about 1.0 ng/ml, alternatively between about 0.8 ng/ml and about 1.0 ng/ml. The first Tₘᵢₙ, which corresponds to the minimum ondansetron concentration (trough level) after the release of the first therapeutically effective amount of ondansetron, may occur between about 1 hour and about 10 hours, alternatively between about 1 hour and 9 hours, alternatively between about 1 hour and 8 hours, alternatively between about 2 hour and 10 hours, alternatively between about 3 hour and 10 hours, alternatively between about 4 hours and 10 hours, alternatively between about 5 hours and 10 hours, alternatively between about 6 hours and 10 hours after administration of the dosage form or pharmaceutical composition.

For the dosage forms and pharmaceutical compositions of this invention, the coating and the second layer both contain therapeutically effective doses of ondansetron. Each of the coating and the second layer may contain about 0.125 to about 1 mg, alternatively about 0.125 to about 0.75 mg, alternatively about 0.2 to about 0.7 mg, alternatively about 0.4 to about 0.6 mg, alternatively about 0.125 mg, alternatively about 0.25 mg, alternatively about 0.4 mg, alternatively about 0.5 mg, alternatively about 0.6 mg, alternatively about 0.75 mg. The coating is formulated for release of ondansetron within about 2 hours, alternatively within about 1 hour, alternatively within about 0.5 hours of administration of the pharmaceutical composition or dosage form. The second layer is formulated to begin releasing ondansetron in not less than 4 hours, alternatively not less than 5 hours, alternatively not less than 6 hours, alternatively not less than 7 hours, alternatively not less than 8 hours, alternatively not less than 9 hours, alternatively not less than 10 hours, alternatively not less than 11 hours, alternatively not less than 12 hours, alternatively not less than 13 hours from the time of administration of the pharmaceutical composition of dosage form. Alternatively, the second layer is formulated for release of ondansetron from the dosage form or pharmaceutical composition within about 6 to about 20 hours, alternatively within about 6 to about 16 hours, alternatively within about 6 to about 14 hours, alternatively within about 6 to about 12 hours following administration of the pharmaceutical composition or dosage form. The first and third layers of the trilayer core may be substantially free of ondansetron.

For the dosage forms and pharmaceutical compositions of this invention, the pharmaceutical composition contains a total dose of ondansetron and the coating (first compartment) and the second layer (second compartment) may contain the same or different therapeutically effective doses of ondansetron. The coating (first compartment) may contain about 30%, alternatively about 40%, alternatively about 50%, alternatively about 60%, alternatively about 70%, alternatively about 80% of the total dose of ondansetron in the pharmaceutical composition. Similarly, the second layer (second compartment) may contain about 30%, alternatively about 40%, alternatively about 50%, alternatively about 60%, alternatively about 70%, alternatively about 80% of the total dose of ondansetron in the pharmaceutical composition.

For any of the compositions or methods described herein, the pharmaceutical composition may also have an in vitro release profile as determined in a USP Type II apparatus with a paddle speed of 50 rpm in water at 37° C. such that about 1 to 50 wt ondansetron is released within about 2 hours, alternatively within about 1 hour, alternatively within about 0.5 hours following initiation of the in vitro release test and about 50 to 100 wt % is released between about 6 hours to about 18 hours, alternatively between about 6 hours to about 14 hours, alternatively between about 6 hours to about 10 hours following initiation of the in vitro release test. Alternatively or in addition to, the composition may have an in vitro release profile comprising two pulses of drug release each having a maxima as determined in a USP Type II apparatus with a paddle speed of 50 rpm in water at 37° C., wherein the maxima of the two pulses of drug release are separated by about 1 hour to about 12 hours, alternatively by about 1 hour to about 10 hours, alternatively by about 2 hour to about 8 hours, alternatively by about 2 hour to about 6 hours, alternatively by about 1 hour, alternatively by about 2 hours, alternatively by about 3 hours, alternatively by about 4 hours, alternatively by about 5 hours, alternatively by about 6 hours, alternatively by about 7 hours, alternatively by about 8 hours, alternatively by about 9 hours, alternatively by about 10 hours, alternatively by about 11 hours, alternatively by about 12 hours.

For any of the compositions or methods described herein, the first and third layers may be substantially free of ondansetron. In one embodiment, the ondansetron contained in the second layer of the core does not leach into the first or third layers of the core during in vitro drug release tests, i.e., the ondansetron does not migrate and subsequently release from the dosage faun due to undesirable mixing of layer contents during the in vitro test. During the dissolution of the first layer, less than 5%, alternatively less than 4%, alternatively less than 3%, alternatively less than 2%, alternatively less than 1% of the total dose of ondansetron (combined amounts from the coating and second layer) is released. Similarly, less than 5%, alternatively less than 4%, alternatively less than 3%, alternatively less than 2%, alternatively less than 1% of the total dose of ondansetron is released between about 2 hours and about 12 hours, alternatively between about 2 hours and about 10 hours, alternatively between about 2 hours and about 8 hours, alternatively between about 2 hours and about 7 hours, alternatively between about 2 hours and about 6 hours following administration of the pharmaceutical composition or dosage form.

For any of the compositions or methods described herein, the patient may be suffering from treatment-resistant OCD, i.e., the patient may not have responded to an SSRI and neuroleptic therapy. Alternatively, the patient may not have responded to an SSRI therapy. Alternatively, the patient may not have yet tried an SSRI therapy or an SSRI and neuroleptic therapy. The ondansetron (or pharmaceutically acceptable salt thereof) could be administered alone, with an SSRI, or with an SSRI and neuroleptic. The present invention could be used for, but is not limited to, OCD patients who have not responded to SSRI therapy or SSRI plus neuroleptic therapy. The present invention could also be used on patients who have not yet tried SSRI therapy or SSRI plus neuroleptic therapy.

For any of the compositions or methods described herein, the ondansetron dosage is administered in a single daily dose. The ondansetron or pharmaceutically acceptable salt thereof can be administered for at least 1 week, alternatively at least 2 weeks, alternatively at least 3 weeks, alternatively at least 4 weeks, alternatively at least 5 weeks, alternatively at least 6 weeks, alternatively at least 7 weeks, alternatively at least 9 weeks, alternatively at least 10 weeks, alternatively at least 11 weeks, alternatively at least 12 weeks, alternatively at least 13 weeks, alternatively at least 14 weeks, alternatively at least 15 weeks, alternatively at least 16 weeks, alternatively at least 1 month, alternatively at least 2 months, alternatively at least 3 months, alternatively at least 4 months, alternatively at least 5 months, alternatively at least 6 months. The ondansetron or pharmaceutically acceptable salt thereof could also be administered daily, alternatively every 2 days, alternatively every 3 days, alternatively every 4 days, alternatively every 5 days, alternatively every 6 days, alternatively every 7 days.

For any of the compositions or methods described herein, suitable salt forms of ondansetron include, but are not limited to, ondansetron hydrochloride, hydrochloride monohydrate, hydrochloride dihydrate, hydrochloride trihydrate, hydrochloride tetrahydrate, citrate, succinate, tartrate, hydrobromide, chloride, bromide, maleate, acetate, benzoate, borate, isethionate, palmetate, oleate, salicylate, besylate, mesylate, tosylate, and sulfate.

### BRIEF DESCRIPTION OF THE DRAWINGS

FIG. 1 shows the cross-section of a capsule.

FIG. 2 shows a bimodal in vitro dissolution release profile for a coated trilayer capsule.

FIG. 3 shows a bimodal in vitro dissolution release profile for a coated trilayer capsule.

FIG. 4 shows an in vitro dissolution release profile for a coated trilayer capsule.

FIG. 5 shows an in vitro dissolution release profile for a coated trilayer capsule.

FIG. 6 shows an in vitro dissolution release profile for a coated trilayer capsule.

FIG. 7 shows an in vitro dissolution release profile for a coated trilayer capsule.

FIG. 8 shows an in vitro dissolution release profile for a coated trilayer capsule.

FIG. 9 shows an in vitro dissolution release profile for a coated trilayer capsule.

FIG. 10 shows an in vitro dissolution release profile for a coated trilayer capsule.

FIG. 11 shows an in vitro dissolution release profile for a coated trilayer capsule.

FIG. 12 shows an in vitro dissolution release profile for a coated trilayer capsule.

FIG. 13 shows an in vitro dissolution release profile for a coated trilayer capsule.

FIG. 14 shows an in vitro dissolution release profile for a coated trilayer capsule.

FIG. 15 shows an in vitro dissolution release profile for a coated trilayer capsule.

FIG. 16 shows an in vitro dissolution release profile for a coated trilayer capsule.

FIG. 17 shows an in vitro dissolution release profile for a coated trilayer capsule.

FIG. 18 shows an in vitro dissolution release profile for a coated trilayer capsule.

FIG. 19 shows an in vitro dissolution release profile for a coated trilayer capsule.

FIG. 20 shows an in vitro dissolution release profile for a coated trilayer capsule.

FIG. 21 depicts the swelling and cracking of the semipermeable membranes after drug release testing.

FIG. 22 depicts drug release profiles for tablets with and without subcoats.

### DETAILED DESCRIPTION OF THE INVENTION

Ondansetron is currently approved for the treatment of nausea and vomiting induced by chemotherapy (commonly described as chemotherapy-induced nausea and vomiting or CINV), and radiation (commonly described as radiation-induced nausea and vomiting or RINV) and nausea and vomiting that is post-operative (commonly described as post-operative nausea and vomiting or POW).

Ondansetron is a highly selective 5-HT₃ receptor antagonist. Anti-emetic effects of ondansetron are mediated via antagonism of 5-hydroxytryptamine receptors located in the chemoreceptor trigger zone in the brain (central), and possibly also on vagal afferents in the upper gastrointestinal tract (peripheral). In animal studies, ondansetron did not appear to have effect on the mesenteric bed or the nerves in the heart suggesting that drug's effects are central rather than peripheral.

### I. EXAMPLES

The following example is offered to illustrate, but not to limit, the claimed invention.

### Example 1

### Ondansetron Pulsatile Dosage Form

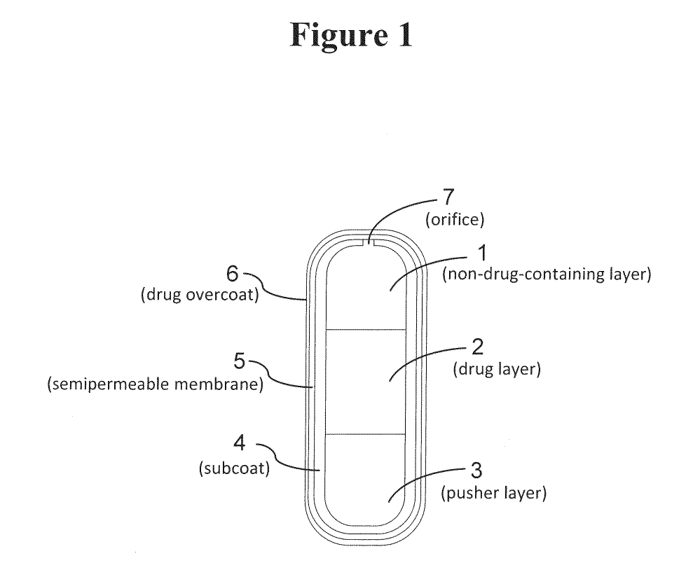

Individuals suffering from OCD could be administered the following ondansetron oral formulation in a coated trilayer osmotic tablet that provides a bimodal release of ondansetron in a 24-hour period. The trilayer osmotic tablet may be in the shape of a capsule that has an outer immediate release coat followed by a semipermeable membrane defining an inner core that contains 3 layers or compartments. As seen in FIG. 1, the tablet contains an inner core with three compartments or layers—first (placebo or non-drug-containing) layer 1, drug-containing layer 2, and pusher layer 3, A semi-permeable membrane 5 having an orifice 7 is generally disposed around, or surrounds, the inner core. An outer drug-containing coat 6 is generally disposed around, or surrounds, the semi-permeable membrane 5. The tablet may also include a subcoat 4, which is located between the inner core and semi-permeable membrane 5.

### The Multi-Layer Tablet

The relative weight percentages (relative to the total weight of the trilayer core) of the first (placebo or non-drug-containing) layer 1, second (drug-containing) layer 2, and third (pusher) layer 3 may be about 25%, about 25%, and about 50%, respectively. Alternatively, the relative weight percentages (relative to the total weight of the trilayer core) of the first layer 1, drug-containing layer 2, and pusher layer may be about 33⅓%, about 33⅓%, and about 33⅓%, respectively. Alternatively, the relative weight percentages (relative to the total weight of the trilayer core) of the first layer 1, drug-containing layer 2, and pusher layer 3 may be between about 25% to about 33⅓%, between about 25% to about 33⅓%, and between about 33⅓% to about 50%, respectively.

The capsule/tablet may contain an inner coating 4 that immediately surrounds (or is generally disposed around) the inner core and serves as a flow promoting layer. The inner coating 4 may include hydroxylpropyl methyl cellulose (HPMC), hydroxypropyl cellulose, hydroxyethyl cellulose, or povidone. Inner coat 4 may be composed of between about 5% to about 15%, alternatively about 1% to about 25%, alternatively about 1% to about 20%, alternatively about 5% to about 20% of the tablet core weight.

The capsule-shaped tablet contains semi-permeable membrane 5 that serves as the rate-controlling membrane. The semi-permeable membrane 5 may include cellulose acetate and a pore-forming copolymer such as polypropylene glycol or polyet poloxamer 188. The ratio of cellulose acetate to polyethylene glycol may be about 99:1 to about 95:5. The ratio of cellulose acetate to poloxamer may be about 90:10 to about 80:20. The amount of the semipermeable membrane ranges from about 5 wt % to about 80 wt % of the core tablet

The outer coat 6 contains ondansetron that can be immediately released after ingestion of the tablet. The outer coat 6 may contain about 0.1 mg to about 2.0 mg, alternatively about 0.1 mg to about 1.8 mg, alternatively about 0.1 mg to about 1.6 mg, alternatively about 0.1 mg to about 1.4 mg, alternatively about 0.2 rug to about 1.4 mg, alternatively about 0.2 mg to about 1.2 mg, alternatively about 0.125 mg, alternatively about 0.5 mg, alternatively about 0.6 mg, alternatively about 0.7 mg, alternatively about 0.8 mg, alternatively about 0.9 mg, alternatively about 1.0 mg, alternatively about 1.1 mg, alternatively about 1.2 mg ondansetron (free base) or its pharmaceutically acceptable salt.

First layer 1 of the inner core is located adjacent to and is in fluid communication with the orifice 7 of the semi-permeable membrane 5. The first layer 1 may comprise suspending polymer (e.g., about 50-80% polyethylene oxide), a disintegrant (e.g., about 2-10 wt % of croscarmellose sodium), a binder (e.g., about 2-8 wt % povidone), an osmotic agent (e.g., about 10-30 wt % sodium chloride), and a lubricant (e.g., about 0.1-1 wt % magnesium stearate) (wt % of the placebo layer). The first layer 1 may be substantially free of ondansetron. It may contain less than 2%, alternatively less than 1%, alternatively less than 0.5%, alternatively less than 0.2%, alternatively less than 0.1%, alternatively less than 0.01% of ondansetron (free base) or its pharmaceutically acceptable salt.

The drug-containing layer 2 (ondansetron-containing layer) is located between (and adjacent to) and in contact with the first layer 1 and pusher layer 3. The drug-containing layer 2 may contain about 0.1 mg to about 2.0 mg, alternatively about 0.1 mg to about 1.8 mg, alternatively about 0.1 mg to about 1.6 mg, alternatively about 0.1 mg to about 1.4 mg, alternatively about 0.2 mg to about 1.4 mg, alternatively about 0.2 mg to about 1.2 mg, alternatively about 0.5 mg, alternatively about 0.6 mg, alternatively about 0.7 mg, alternatively about 0.8 mg, alternatively about 0.9 mg, alternatively about 1.0 mg, alternatively about 1.1 mg, alternatively about 1.2 mg of ondansetron (free base) or its pharmaceutically acceptable salt. In one embodiment, the drug-containing layer 2 may contain about 0.2 to about 2.4 wt % ondansetron or its pharmaceutically acceptable salt, a disintegrant (e.g., about 2-10 wt % of croscarmellose sodium), an osmotic agent (e.g., about 10-30 wt % of sodium chloride), a binder (e.g., about 2-8 wt % povidone), a glidant (e.g., about 0.5-4 wt % silicon dioxide), a lubricant (e.g., about 0.1-1 wt % magnesium stearate), an antioxidant (e.g., about 0.01-1 wt % of butylated hydroxy toluene), and a suspending polymer (e.g., about 50-80 wt % polyethylene oxide) (wt % of the drug-containing layer).

The expandable pusher layer 3 is located adjacent to and in contact with the ondansetron-containing layer 2. The pusher layer 3 may contain an osmotic agent (e.g., about 10 wt % to about 30 wt % sodium chloride), a binder (e.g., about 2 wt % to about 10 wt % povidone), a glidant (e.g., about 0.5 wt % to about 4 wt % silicon dioxide), a lubricant (e.g., about 0.1 wt % to about 2 wt % magnesium stearate), and a suspending polymer (e.g., about 60 wt % to about 80% polyethylene oxide) (wt % of the total pusher layer). The expandable pusher layer 3 may be substantially free of ondansetron. It may contain less than 2%, alternatively less than 1%, alternatively less than 0.5%, alternatively less than 0.2%, alternatively less than 0.1%, alternatively less than 0.01% ondansetron (free base) or its pharmaceutically acceptable salt. The expandable pusher layer 3 may account for 33% or more, alternatively 40% or more, alternatively 50% or more, alternatively 60% or more, alternatively 70% or more of the total weight of the trilayer osmotic core.

The polyethylene oxide described above may have average molecular weights between 50K and 9 million. For the placebo layer 1, the polyethylene oxide may have an average molecular weight of about 200K, alternatively about 300K, alternatively about 400K, alternatively about 500K, alternatively about 600K, alternatively about 700K, alternatively about 800K. For the drug-containing layer 2, the polyethylene oxide may have an average molecular weight of about 50K, alternatively about 100K, alternatively about 200K, alternatively about 300K, alternatively about 400K, alternatively about 500K. For the expandable push layer 3, the polyethylene oxide may have an average molecular weight of about 2 million, alternatively about 3 million, alternatively about 4 million, alternatively about 5 million, alternatively about 6 million, alternatively about 7 million, alternatively about 8 million, alternatively about 9 million.

Table 1 lists examples of compositions of trilayer tablets that provide a bimodal release of ondansetron. The trilayer osmotic tablets have an outer immediate release coat containing ondansetron followed by a semipermeable membrane defining an inner core that contains 3 layers or compartments, wherein one of the layers contains ondansetron,

TABLE 1
Nominal composition of LCT Ondandsetron trilayer tablet

| | LCT040965- | LCT040965- | LCT040965- | LCT040965- | LCT040965- |
|---|---|---|---|---|---|
| Components | 14F25% | 14L20% | 14L20%A | 14F20% | 14G20% |
| First layer | | | | | |
| a. Polyethylene Oxide, 300K | 35 | 35 | 35 | 35 | 35 |
| b. Povidone (K29-32) | 2.5 | 2.5 | 2.5 | 2.5 | 2.5 |
| c. Croscarmellose Na | 2 | 2 | 2 | 2 | 2 |
| d. Sodium Chloride | 10 | 10 | 10 | 10 | 10 |
| e. Magnesium Stearate | 0.25 | 0.25 | 0.25 | 0.25 | 0.25 |
| f. Ferric oxide | 0.25 | 0.25 | 0.25 | 0.25 | 0.25 |
| Total weight of first layer (mg) | 50.0 | 50.0 | 50.0 | 50.0 | 50.0 |
| Second layer | | | | | |
| a. Ondansetron Hydrochloride | 0.9 | 0.75 | 0.75 | 0.9 | 0.9 |
| b. Polyethylene Oxide, 100K | 31.8 | 31.95 | 31.95 | 31.8 | 31.8 |
| c. Povidone (K29-32) | 2.5 | 2.5 | 2.5 | 2.5 | 2.5 |
| d. Croscarmellose Na | 3 | 3 | 3 | 3 | 3 |
| e. Silicon Dioxide (Sylloid 244FP) | 1 | 1 | 1 | 1 | 1 |
| f. Sodium Chloride | 10 | 10 | 10 | 10 | 10 |
| g. Magnesium Stearate | 0.25 | 0.25 | 0.25 | 0.25 | 0.25 |
| h. Butylated Hydroxytoluene | 0.025 | 0.025 | 0.025 | 0.025 | 0.025 |
| i. Ferric oxide | 1 | 1 | 1 | 1 | 1 |
| Total weight of second layer (mg) | 50.5 | 50.5 | 50.5 | 50.5 | 50.5 |
| Pusher layer | | | | | |
| a. Polyethylene Oxide, 7000K | 72.45 | 72.45 | 72.45 | 72.45 | 72.45 |
| b. Povidone (K29-32) | 5 | 5 | 5 | 5 | 5 |
| c. Silicon Dioxide (Sylloid 244FP) | 1.33 | 1.33 | 1.33 | 1.33 | 1.33 |
| d. Sodium Chloride | 20 | 20 | 20 | 20 | 20 |
| e. Magnesium Stearate | 0.5 | 0.5 | 0.5 | 0.5 | 0.5 |
| f. Butylated Hydroxytoluene | 0.05 | 0.05 | 0.05 | 0.05 | 0.05 |
| g. Ferric oxide (yellow) | 0.67 | 0.67 | 0.67 | 0.67 | 0.67 |
| Total weight of push layer (mg) | 100.0 | 100.0 | 100.0 | 100.0 | 100.0 |
| Subcoat | | | | | |
| a. Hydroxypropylmethyl Cellulose | 11.84 | 9.35 | 9.35 | 10.54 | 9.78 |
| Total weight of subcoat layer (mg) | 11.84 | 9.35 | 9.35 | 10.54 | 9.78 |
| Rate controlling membrane | | | | | |
| a. Cellulose Acetate (398-10) mg | 45.8 | 35.0 | 35.0 | 36.8 | 34.3 |
| b. Polyoxypropylene-polyoxyethylene | 4.0 | 5.7 | 5.7 | 3.2 | 6.5 |
| Block Copolymer (Lutrol F68) | | | | | |
| Total weight of rate controlling | 49.8 | 40.7 | 40.7 | 40.0 | 40.8 |
| membrane (mg) | | | | | |
| Immediate release overcoat | | | | | |
| a. Ondansetron Hydrochloride | 0.6 | 0.5 | 0.6 | 0.6 | 0.6 |
| b. Hydroxypropylmethyl Cellulose | 20.0 | 20.0 | 20.0 | 20.0 | 20.0 |
| Total weight of immediate release | 20.6 | 20.5 | 20.6 | 20.6 | 20.6 |
| overcoat (mg) | | | | | |
| Tablet wt (mg) | 282.7 | 271.0 | 271.1 | 271.6 | 271.6 |
| Lag Time (h) | 9.0 | 7.0 | 7.0 | 6.0 | 5.5 |
| Drug Ratio (immediate release/ | 0.4/0.6 | 0.4/0.6 | 0.5/0.6 | 0.4/0.6 | 0.4/0.6 |
| controlled release) | | | | | |

The capsule-shaped tablet contains a therapeutically effective dose of ondansetron that is released in a bimodal pharmacokinetic profile. It is designed for once-a-day administration to deliver ondansetron for about 24 hours utilizing a capsule-shaped trilayer tablet. As explained above, the capsule-shaped trilayer tablet contains a dose of ondansetron in at least two separate compartments/layers of the tablet. The total dose of ondansetron (D) for the tablet is the sum of the doses contained in each of the layers or compartments, i.e., the sum of doses contained in the outer coating of ondansetron and in the drug-containing layer in the inner core. The capsule-shaped trilayer tablet containing ondansetron or a pharmaceutically acceptable salt has a pulsatile release profile of the total dose of ondansetron that provides a bimodal distribution of plasma ondansetron concentration with first and second Cₘₐₓ's, which occur at first and second Tₘₐₓ's, respectively. The first dose-normalized Cₘₐₓ (i.e., normalized for a 1 mg total dose in the tablet) may be about 0.1 ng/ml to about 4.0 ng/ml, alternatively about 0.2 ng/ml to about 3.5 ng/ml, alternatively about 0.4 ng/ml to about 3.5 ng/ml, alternatively about 0.6 rig/ml to about 3.5 ng/ml, alternatively about 0.7 ng/ml to about 3.5 ng/ml. The first Tₘₐₓ may occur within about 0.1 hours to about 5 hours, alternatively within about 0.1 to about 4.5 hours, alternatively within about 0.1 to about 4 hours, alternatively within about 0.1 to about 3.5 hours, alternatively within about 0.1 to about 3 hours, alternatively within about 0.1 to about 2.5 hours, alternatively within about 0.1 to about 2 hours, alternatively within about 0.1 to about 1.5 hours, alternatively within about 0.1 to about 1 hour. Similarly, the second dose-normalized Cₘₐₓ (i.e., normalized for a 1 mg total dose in the tablet) may be about 0.1 ng/ml to about 4.0 ng/ml, alternatively about 0.2 ng/ml to about 3.5 ng/ml, alternatively about 0.4 ng/ml to about 3.5 ng/ml, alternatively about 0.6 ng/ml to about 3 ng/ml, alternatively about 0.7 ng/ml to about 2.5 ng/ml. The second Tₘₐₓ may occur within about 6 hours to about 20 hours, alternatively within about 7 hours to about 18 hours, alternatively within about 8 hours to about 16 hours, alternatively within about 9 hours to about 15 hours, alternatively within about 10 hours to about 14 hours. Additionally, the two ondansetron plasma peak maxima may be separated by from about 1 hour to about 12 hours, alternatively from about 2 hours to about 12 hours, alternatively from about 3 hours to about 11 hours, alternatively from about 4 hours to about 10 hours, alternatively from about 5 hours to about 9 hours, alternatively from about 6 hours to about 8 hours, alternatively by about 5 hours, alternatively by about 6 hours, alternatively by about 7 hours, alternatively by about 8 hours, alternatively by about 9 hours, alternatively by about 10 hours, alternatively by about 11 hours, alternatively by about 12 hours, alternatively by about 13 hours, alternatively by about 14 hours, alternatively by about 15 hours.

This bimodal profile provides therapeutic delivery above the minimum effective concentration while keeping the plasma levels below the maximum tolerated concentration and low enough such that side effects will be reduced. This delivery profile provides 24 hours of efficacy without initially undesirable high plasma levels.

### Example 2

### In Vitro Dissolution Test

### Sample Preparation

Tablets were dropped into 500 mL of de-aerated de-ionized water (DI H₂O), 37° C. using USP dissolution apparatus 2 (paddles) at 50 rpm. Samples were collected and transferred to HPLC vials at 6, 8, 9, 10, 11, 12, 13, 14, 15, 16, 17, 18, 20, and 2.4 hours,

### HPLC Analysis

HPLC analysis was performed on a Waters 2695 HPLC system (SN B00SD6 021M/G07SHC442A) equipped with a Waters 2996 PDA detector (SN E06296 830M) or Waters 2487 dual wavelength absorbance detector (SN G07487681M). A Zorbax SB-CN, 3 μm, 4.5×50 mm column was used (SN USJF001656). The mobile phase consisted of 45% Methanol and 55% of a 25 mM KH₂PO₄ pH=6.3 buffer. Samples were analyzed at 250 nm, 100 μl injections, with a flow rate of 10.0 mL/min.

In the in vitro dissolution test, the coated trilayer osmotic tablet was adapted to release the ondansetron at rates such that about 20 wt % to about 70 wt % of the total ondansetron dose was released over a time ranging from about 0 hours to about 1 hour, followed by release of about 0 wt % to about 20 wt % of the total ondansetron dose from about 1 hour to about 4 hours, and about 30 wt % to about 90 wt of the total ondansetron dose over a time ranging from about 4 hours to 23 hours. The lag time is the estimated time period after the drug release from the drug overcoat is complete and less than 5% drug is released from the drug-containing layer within the core.

### Example 3

### Coated Trilayer Tablet Release Profile (8 Hour Lag Time)

The trilayer tablet contains 0.5 mg Ondansetron.HCl in the immediate release overcoat and 0.75 mg Ondansetron.HCl in the internal drug layer (second layer).

The trilayer core was prepared as follows. The first layer (placebo layer) included 4% croscarmellose sodium, 5% povidone (K-29-32), 20% sodium chloride, 0.5% magnesium stearate, 0.5% iron oxide color, and 70% polyethylene oxide 300K average molecular weight. All ingredients were screened separately through a 30 mesh screen then dry blended excluding magnesium stearate in a conventional blender for 13 minutes followed by the addition of magnesium stearate with the blending continued for another 5 minutes to yield a homogenous blend. The second layer (drug layer) included 1.5% ondansetron.HCl, 6.0% croscarmellose sodium, 0.05% butylated hydroxytoluene, 20% sodium chloride, 5.0% povidone (K-29-32), 2.0% sylloid 244 FP, 0.5% magnesium stearate, 2% iron oxide color, and 63.3% polyethylene oxide 100K average molecular weight. The ingredients were screened and blended identically as the first layer described above. The third layer (pusher layer) included 0.05% butylated hydroxytoluene, 20% sodium chloride, 5% povidone (K-29-32), 1.33% sylloid 244 FP, 0.5% magnesium stearate, 0.67% iron oxide color, and 72.45% polyethylene oxide 7 million average molecular weight. The ingredients were screened and blended identically as the non-drug containing (placebo) and drug layers described above 50 mg of the non-drag containing (placebo) layer, 50 mg of the drug layer, and 100 mg of the pusher layer in this order were added into a 6/32 inch (4.76 cm) diameter punch-die set and tamped in between each layer. The three layers were then compressed under a pressure head of 500 psi using a Carver press.

The trilayer core was coated with a polymer coating. This subcoat was prepared from a 10% aqueous solution of hydroxypropylmethylcellulose (HPMC). The HPMC solution was sprayed onto the trilayer core to provide a 10 mg subcoat layer (5% weight gain of the core). The coated tablet with HPMC was dried for 1 hour at 48° C.

The trilayer tablet was manufactured into a dosage form by coating it with an additional layer of polymer coating. This second coating layer (semipermeable membrane) included 99:1 wt ratio of cellulose acetate and polyethylene glycol 3350, which was dissolved in a co-solvent of acetone and water in 95:5 wt:wt composition to make a 4% solid solution. This composition was sprayed onto and around the HPMC-coated trilayer core to provide a 40.0 mg semipermeable wall (20.0% weight gain of the core). The semipermeable-walled trilayer tablet was drilled to provide a 20 mil (0.51 mm) orifice to contact the non-drug containing (placebo) layer and the exterior of the dosage form. The residual solvent was removed by drying for 72 hours at 45° C. and 45% relative humidity. The dosage form was then dried further for 6 hours at 45° C. to remove excess moisture.

The trilayer tablet was further coated with an exterior coating of ondansetron hydrochloride. A solution was sprayed onto the trilayer tablet to provide a 10 mg HPMC and 0.5 mg ondansetron hydrochloride. The coated tablet was dried for 1 hour at 48° C.

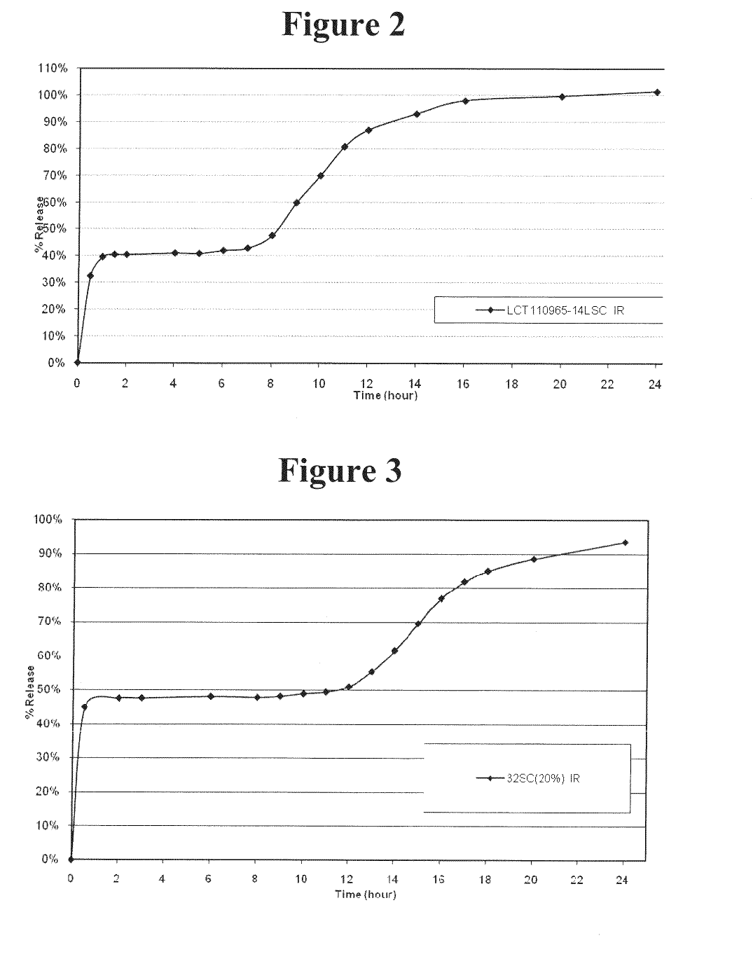

The in vitro release profile (see Example 9 for description of method) is shown in FIG. 2. Approximately 30% to 45% of the total dosage of ondansetron is released from about 30 minutes to about 8 hours after the start of the in vitro release test. The remaining approximately 45% to 100% of the total dosage of ondansetron is released from about 8 hours to about 18 hours after the start of the in vitro release test.

### Example 4

### Coated Trilayer Tablet Release Profile (12 Hour Lag Time—LCT 0509 65-32SC 20%—99:1 CA:PEG)

The trilayer tablet contains 0.6 mg Ondansetron.HCl in the immediate release overcoat and 0.6 mg Ondansetron.HCl in the internal drug layer (second layer).

The trilayer core was prepared as follows. The first layer (placebo layer) included 4% croscarmellose sodium, 5% povidone (K-29-32), 20% sodium chloride, 0.5% magnesium stearate, 1% iron oxide color, and 69.5% polyethylene oxide 1 million average molecular weight. All ingredients were screened separately through a 30 mesh screen then dry blended excluding magnesium stearate in a conventional blender for 13 minutes followed by the addition of magnesium stearate with the blending continued for another 5 minutes to yield a homogenous blend. The second layer (drug layer) included 1.2% ondansetron HCl, 10% croscarmellose sodium, 0.06% butylated hydroxytoluene, 30% sodium chloride, 5.0% povidone (K-29-32), 2.0% sylloid 244 FP, 0.5% magnesium stearate, 1% iron oxide color, and 50.24% polyethylene oxide 100K molecular weight. The ingredients were screened and blended identically as the placebo layer described above. The third layer (pusher layer) included 0.05% butylated hydroxytoluene, 20% sodium chloride, 5% povidone (K-29-32), 1.33% sylloid 244 FP, 0.5% magnesium stearate, 0.67% iron oxide color, and 72.45% polyethylene oxide 7 million average molecular weight. The ingredients were screened and blended identically as the first (placebo) and drug layers described above 50 mg of the first (Placebo) layer, 50 mg of the drug layer, and 100 mg of the pusher layer in this order was added into a 6/32 inch (4.76 cm) diameter punch-die set and tamped in between each layer. The three layers were then compressed under a pressure head of 500 psi using a carver press.

The trilayer tablet was coated with a polymer coating. This subcoat was prepared from a 10% aqueous solution of hydroxypropylmethylcellulose (HPMC). The solution was sprayed onto the trilayer core described above to provide a 10 mg subcoat layer (5% weight gain of the core). The coated tablet with HPMC was dried for 1 hour at 48′C.

The trilayer tablet coated with the polymer coating described above was then coated with another layer of polymer coating. This second coating layer formed the semipermeable membrane and was prepared with a 99:1 wt ratio of cellulose acetate and polyethylene glycol 3350, which was dissolved in a co-solvent of acetone and water in 95:5 wt:wt composition to make a 4% solid solution. The composition was sprayed onto and around the HPMC-coated trilayer core to provide a 40 mg semipermeable membrane/wall (20% weight gain of the core). Next, the semipermeable walled, trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage forms were then dried further for 6 hours at 45° C. to remove excess moisture.

The dosage form that was prepared above containing a total ondansetron dosage of 1.2 mg ondansetron salt. As seen in FIG. 3, about 45% of the total ondansetron dose (about 0.54 mg) was released within the first 30 minutes of the in vitro dissolution test and up to about 0.6 mg between 0 and 12 hours. About 1.12 mg of ondansetron was released within 0 to 24 hours (93.4% drug release at 24 hours). The lag time prior to the initiation of the release of ondansetron from the second layer was about 12 hours.

### Example 5

### Trilayer Tablet Release Profile (Core—LCT 0409 65-14)

A therapeutic ondansetron trilayer tablet core was prepared as follows. The first layer (placebo layer) included 4% croscarmellose sodium, 5% povidone (K-29-32), 20% sodium chloride, 0.5% magnesium stearate, 1% iron oxide color, and 69.5% polyethylene oxide 300K average molecular weight. All ingredients were screened separately through a 30 mesh screen then dry blended excluding magnesium stearate in a conventional blender for 13 minutes followed by the addition of magnesium stearate with the blending continued for another 5 minutes to yield a homogenous blend. The second layer (drug layer) included 1.8% ondansetron HCl, 6.0% croscarmellose sodium, 0.05% butylated hydroxytoluene, 20% sodium chloride, 5.0% povidone (K-29-32), 2.0% sylloid 244 FP, 0.5% magnesium stearate, 1% iron oxide color, and 63.6% polyethylene oxide 100K average molecular weight. The ingredients were screened and blended identically as the first (placebo) layer described above. The third layer (pusher layer) included 0.05% butylated hydroxytoluene, 20% sodium chloride, 5% povidone (K-29-32), 1.33% sylloid 244 FP, 0.5% magnesium stearate, 0.67% iron oxide color, and 72.45% polyethylene oxide 7 million average molecular weight. The ingredients were screened and blended identically as the first (placebo) and drug layers described above 50 mg of the first (placebo) layer, 50 mg of the drug layer, and 100 mg of the push layer in this order was added into a 6/32 inch (4.76 cm) diameter punch-die set and tamped in between each layer. The three layers were then compressed under a pressure head of 500 psi using a Carver press.

The trilayer tablet was coated with a polymer coating. This subcoat was prepared from a 10% of aqueous solution of hydroxypropylmethylcellulose (HPMC). The solution was sprayed onto the trilayer cores as prepared above to provide a 10 mg subcoat layer (5% weight gain of the core). The coated tablet with HPMC was dried for 1 hour at 48° C.

### Example 6

### Trilayer Tablet Release Profile (3 Hour Lag Time—LCT 0409 65-14G SC 15.1% —84:10 CA:Lutrol)

The trilayer core as described in Example 5 was manufactured into a dosage form by being coated with another layer of polymer coating. This second coating layer (semipermeable membrane) included 84:16 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC coated trilayer cores to provide a 30.2 mg semipermeable wall (15.1% weight gain of the core). Next, the semipermeable-walled, trilayer tablet was drilled to provide a 20 mil (0.51 mm) orifice to contact the first (placebo) layer and the exterior of the dosage form. The residual solvent was removed by drying for 72 hours at 45° C. and 45% relative humidity. Next, the dosage forms were dried further for 6 hours at 45° C. to remove excess moisture.

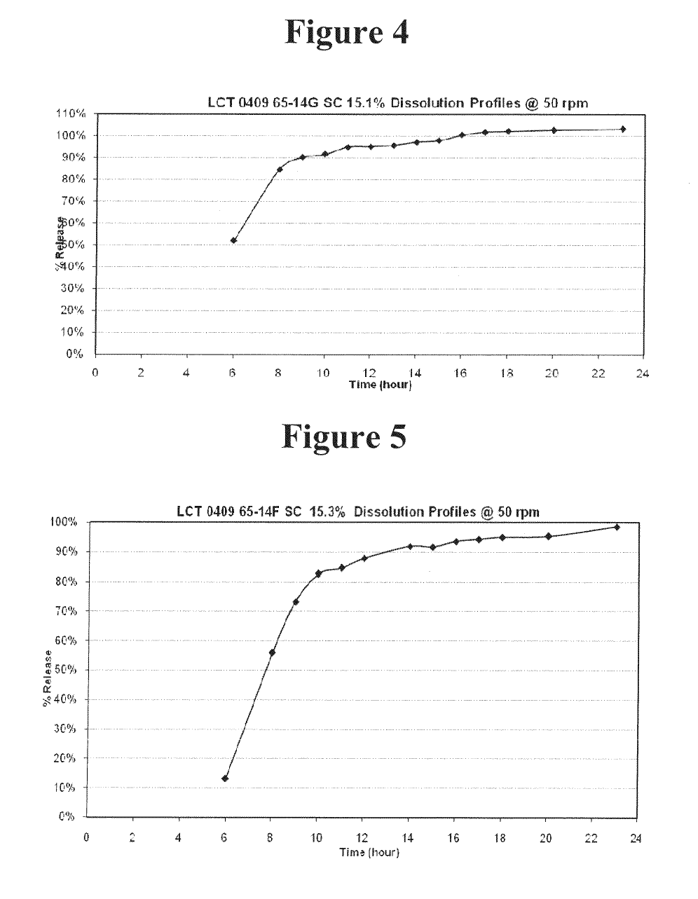

The dosage form contained 0.9 mg ondansetron salt. As seen in FIG. 4, the dosage form released 0.47 mg in 0 to 6 hours, up to 0.76 mg in 0 to 8 hours, up to 0.83 mg in 0 to 10 hours, up to 0.86 mg in 0 to 12 hours, up to 0.88 mg in 0 to 14 hours, up to 0.91 mg in 0 to 16 hours, up to 0.92 mg in 0 to 18 hours, up to 0.93 in 0 to 20 hours, and up to 0.93 mg in 0 to 23 hours. The lag time was 3 hours, reached 90% drug release at 9 hours, had a second pulse time (time interval between start to 90% of drag release) of 6 hours, and a slope of 0.21 during the drug release from 5% to 70%.

### Example 7

### Trilayer Tablet Release Profile (5 Hour Lag Time—LCT 0409 65-14F SC 15.3% —88:12 CA:Lutrol)

The trilayer core as described in Example 5 was manufactured into a dosage form by being coated with another layer of polymer coating. This second coating layer (semipermeable membrane) included 88:12 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC-coated trilayer core to provide a 30.6 mg semipermeable wall (15.3% weight gain of the core). Next, the semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

The dosage form contained 0.9 mg ondansetron salt. As seen in FIG. 5, the dosage form released 0.12 mg ondansetron in 0 to 6 hours, up to 0.50 mg in 0 to 8 hours, up to 0.74 mg in 0 to 10 hours, up to 0.79 mg in 0 to 12 hours, up to 0.83 mg in 0 to 14 hours, up to 0.84 mg in 0 to 16 hours, up to 0.85 mg in 0 to 18 hours, up to 0.86 in 0 to 20 hours, and up to 0.89 mg in 0 to 23 hours. The lag time was 5 hours, reached 90% drug release at 13 hours, had a second pulse time (time interval between start to 90% of drug release) of 8 hours, and a slope of 0.20 during the drug release from 5% to 70%.

### Example 8

### Trilayer Tablet Release Profile (5 Hour Lag Time—LCT 0409 65-14G SC 20.4% —84:16 CA:Lutrol)

The trilayer core as described in Example 5 was manufactured into a dosage form by being coated with another layer of polymer coating. This second coating layer (semipermeable membrane) included 84:16 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC-coated trilayer core to provide a 40.8 mg semipermeable wall (20.4% weight gain of the core). Next, the semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

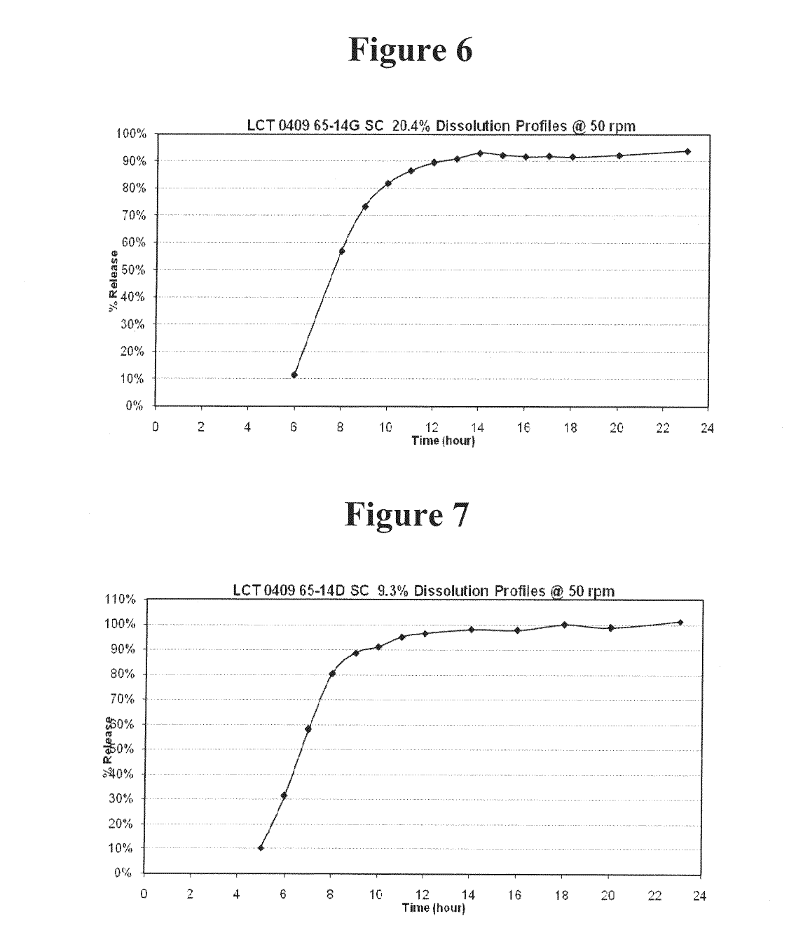

The dosage form contained 0.9 mg ondansetron salt. As seen in FIG. 6, the dosage form released 0.10 mg ondansetron in 0 to 6 hours, up to 0.51 mg in 0 to 8 hours, up to 0.74 mg in 0 to 10 hours, up to 0.81 mg in 0 to 12 hours, up to 0.84 mg in 0 to 14 hours, up to 0.83 mg in 0 to 16 hours, up to 0.83 mg in 0 to 18 hours, up to 0.83 in 0 to 20 hours, and up to 0.85 mg in 0 to 23 hours. The lag time was 5 hours, reached 90% drug release at 12 hours, had a second pulse time (time interval between start to 90% of drug release) of 7 hours, and a slope of 0.21 during the drug release from 5% to 70%.

### Example 9

### Trilayer Tablet Release Profile (5 Hour Lag Time—LCT 0409 65-14D SC 9.3%—99:1 CA:PEG)

The trilayer core as described in Example 5 was manufactured into a dosage form by being coated with another layer of polymer coating. This second coating layer (semipermeable membrane) includes 99:1 wt ratio of cellulose acetate and polyethylene glycol 3350, which was dissolved in a cosolvent of acetone and water in 95:5 wt:wt composition to make a 4% solid solution. This composition was sprayed onto and around the HPMC-coated trilayer core to provide an 18.6 mg semipermeable wall (91% weight gain of the core). Next, the semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. Next, the dosage forms were dried further for 6 hours at 45° C. to remove excess moisture.

The dosage form contained 0.9 mg ondansetron salt. As seen in FIG. 7, the dosage form released 0.09 mg of ondansetron in 0 to 5 hours, up to 0.28 mg in 0 to 6 hours, up to 0.72 mg in 0 to 8 hours, up to 082 mg in 0 to 10 hours, up to 0.87 mg in 0 to 12 hours, up to 0.88 mg in 0 to 14 hours, up to 0.88 mg in 0 to 16 hours, up to 0.90 mg in 0 to 18 hours, up to 0.89 in 0 to 20 hours, and up to 0.91 mg in 0 to 23 hours. The lag time was 5 hours, reached 90% drug release at 9 hours, had a second pulse time (time interval between start to 90% of drug release) of 4 hours, and a slope of 0.24 during the drug release from 5% to 70%.

### Example 10

### Trilayer Tablet Release Profile (6 Hour Lag Time—LCT 0409 65-14E SC 1.43%—92:8 CA:Lutrol)

The trilayer core as described in Example 5 was manufactured into a dosage form by being coated with another layer of polymer coating. This second coating layer (semipermeable membrane) includes 92:8 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC-coated trilayer core to provide a 28.5 mg semipermeable wall (14.3% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

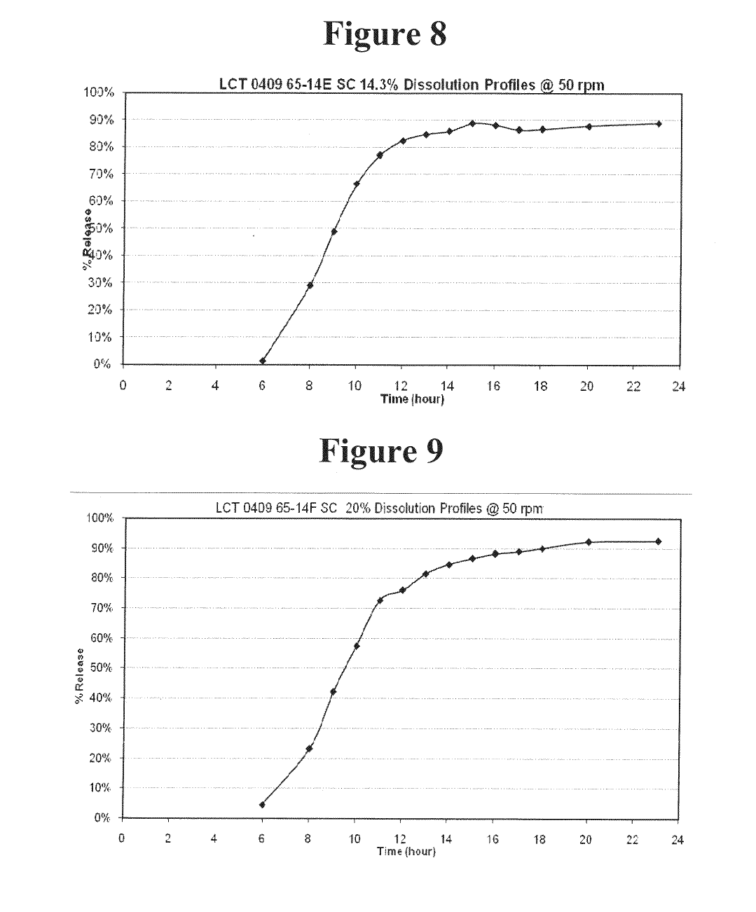

The dosage contained 0.9 mg ondansetron salt. As seen in FIG. 8, the dosage form released 0.01 mg of ondansetron in 0 to 6 hours, up to 0.26 mg in 0 to 8 hours, up to 0.60 mg in 0 to 10 hours, up to 0.74 mg in 0 to 12 hours, up to 0.77 mg in 0 to 14 hours, up to 0.79 mg in 0 to 16 hours, up to 0.78 mg in 0 to 18 hours, up to 0.79 in 0 to 20 hours, and up to 0.80 mg in 0 to 23 hours. The lag time was 6 hours, reached 90% drug release at 15 hours, had a second pulse time (time interval between start to 90% of drug release) of 8 hours, and a slope of 0.16 during the drug release from 5% to 70%.

### Example 11

### Trilayer Tablet Release Profile (6 Hour Lag Time—LCT 0409 65-14F SC 20% —88:12 CA:Lutrol)

The trilayer core as described in Example 5 was manufactured into a dosage form by being coated with another layer of polymer coating. This second coating layer (semipermeable membrane) included 88:12 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC-coated trilayer core to provide a 40 mg semipermeable wall (20% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

The dosage form contained 0.9 mg ondansetron salt. As seen in FIG. 9, the dosage form released 0.04 mg of ondansetron in 0 to 6 hours, up to 0.21 mg in 0 to 8 hours, up to 0.52 mg in 0 to 10 hours, up to 0.68 mg in 0 to 12 hours, up to 0.76 mg in 0 to 14 hours, up to 0.79 mg in 0 to 16 hours, up to 0.81 mg in 0 to 18 hours, up to 083 in 0 to 20 hours, and up to 0.83 mg in 0 to 23 hours. The lag time was 6 hours, reached 90% drug release at 16 hours, had a second pulse time (time interval between start to 90% of drug release) of 10 hours, and a slope of 0.16 during the drug release from 5% to 70%.

### Example 12

### Trilayer Tablet Release Profile (6 Hour Lag Time—LCT 0409 65-14G SC 24.9%—84:16 CA:Lutrol)

The trilayer core as described in Example 5 was manufactured into a dosage form by being coated with another layer of polymer coating. This second coating layer (semipermeable membrane) included 84:16 wt ratio of cellulose acetate and copolymer of polyethylene oxide and polypropylene oxide (Lutrol) which was dissolved in a cosolvent of acetone and water in 95:5 wt:wt composition to make a 4% solid solution. This composition was sprayed onto and around the HPMC coated trilayer cores to provide a 49.8 mg semipermeable wall (24.9% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage forms were then dried further for 6 hours at 45° C. to remove excess moisture.

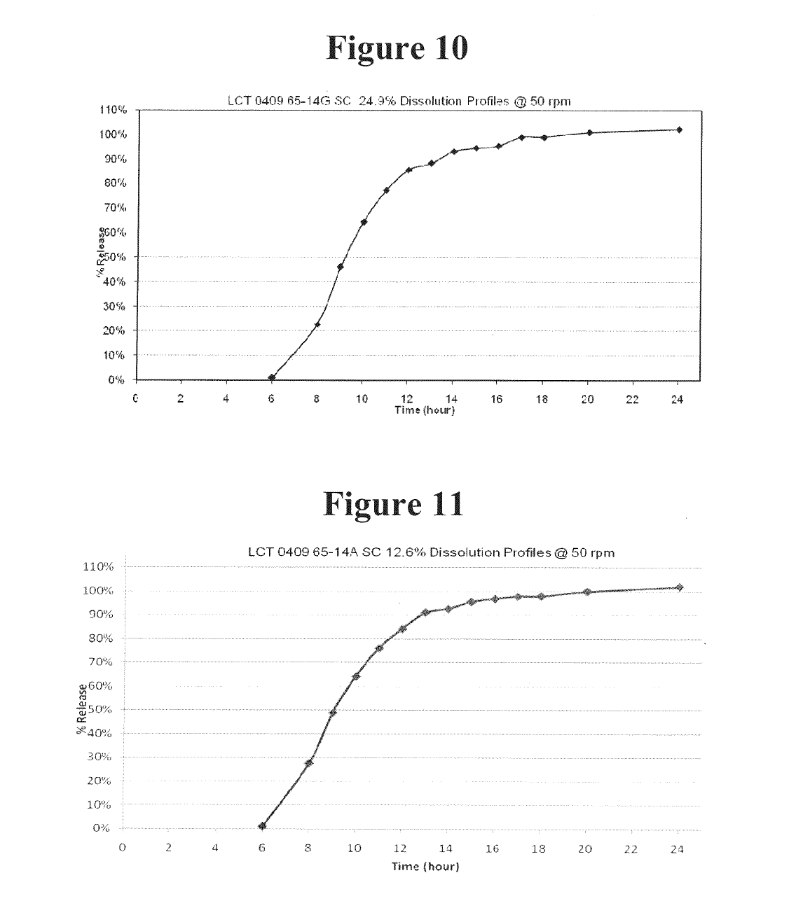

The dosage form contained 0.9 mg ondansetron salt. As seen in FIG. 10, the dosage form released 0.01 mg of ondansetron in 0 to 6 hours, up to 0.21 mg in 0 to 8 hours, up to 0.58 mg in 0 to 10 hours, up to 0.77 mg in 0 to 12 hours, up to 0.84 mg in 0 to 14 hours, up to 0.86 mg in 0 to 16 hours, up to 0.89 mg in 0 to 18 hours, up to 0.91 in 0 to 20 hours, and up to 0.92 mg in 0 to 24 hours. The lag time was 6 hours, reached 90% drug release at 13 hours, had a second pulse time (time interval between start to 90% of drug release) of 7 hours, and a slope of 0.18 during the drug release from 5% to 70%.

### Example 13

### Trilayer Tablet Release Profile (6 Hour Lag Time—LCT 0409 65-14A SC 12.6%—99:1 CA:PEG)

The trilayer core as described in Example 5 was manufactured into a dosage form by being coated with another layer of polymer coating. This second coating layer (semipermeable membrane) includes 99:1 wt ratio of cellulose acetate and polyethylene glycol 3350, which was dissolved in a cosolvent of acetone and water in 95:5 wt:wt composition to make a 4% solid solution. This composition was sprayed onto and around the HPMC-coated trilayer core to provide a 25.2 mg semipermeable wall (12.6% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

The dosage form contained 0.9 mg ondansetron salt. As seen in FIG. 11, the dosage form released 0.01 mg ondansetron in 0 to 6 hours, up to 0.25 mg in 0 to 8 hours, up to 0.58 mg in 0 to 10 hours, up to 0.76 mg in 0 to 12 hours, up to 0.83 mg in 0 to 14 hours, up to 0.87 mg in 0 to 16 hours, up to 0.88 mg in 0 to 18 hours, up to 0.90 in 0 to 20 hours, and up to 0.92 mg in 0 to 24 hours. The lag time was 6 hours, reached 90% drug release at 13 hours, had a second pulse time (time interval between start to 90% of drug release) of 7 hours, and a slope of 0.14 during the drug release from 5% to 70%.

### Example 14

### Trilayer Tablet Release Profile (7 Hour Lag Time—LCT 0409 65-14D SC 139%—99:1 CA:PEG)

The trilayer core as described in Example 5 was manufactured into a dosage form by being coated with another layer of polymer coating. This second coating layer (semipermeable membrane) includes 99:1 wt ratio of cellulose acetate and polyethylene glycol 3350, which was dissolved in a cosolvent of acetone and water in 95:5 wt:wt composition to make a 4% solid solution. This composition was sprayed onto and around the HPMC coated trilayer cores to provide a 27.8 mg semipermeable wall (13.9% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

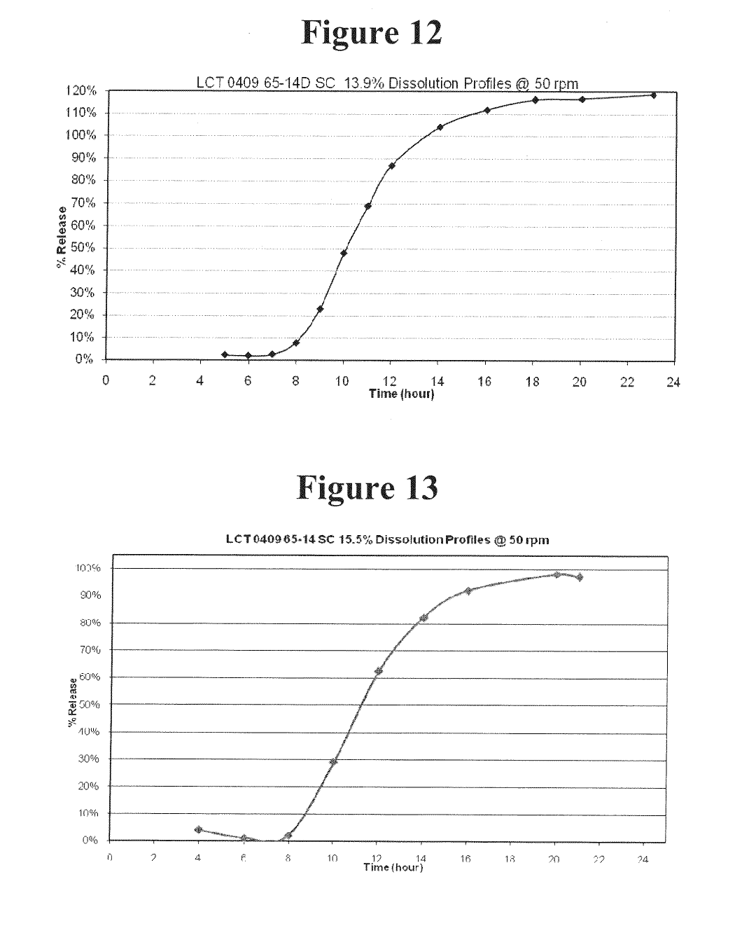

The dosage form contained 0.9 mg ondansetron salt. As seen in FIG. 12, the dosage form released 0.02 mg of ondansetron in 0 to 5 hours, up to 0.02 mg in 0 to 6 hours, up to 0.07 mg in 0 to 8 hours, up to 0.43 mg in 0 to 10 hours, up to 0.78 mg in 0 to 12 hours, up to 0.94 mg in 0 to 14 hours, up to 1.01, mg in 0 to 16 hours, up to 1.05 mg in 0 to 18 hours, up to 1.05 mg in 0 to 20 hours, and up to 1.07 mg in 0 to 23 hours. The lag time was 7 hours, reached 90% drug release at 12 hours, had a second pulse time (time interval between start to 90% of drug release) of 5 hours, and a slope of 0.21 during the drug release from 5% to 70%.

### Example 15

### Trilayer Tablet Release Profile (8 Hour Lag Time—LCT 0409 65-14 SC 15.5%—99:1 CA:PEG)

The trilayer core as described in Example 5 was manufactured into a dosage form by being coated with another layer of polymer coating. This second coating layer (semipermeable membrane) includes 99:1 wt ratio of cellulose acetate and polyethylene glycol 3350, which was dissolved in a cosolvent of acetone and water in 95:5 wt:wt composition to make a 4% solid solution. This composition was sprayed onto and around the HPMC-coated trilayer core to provide a 31.0 mg semipermeable wall (15.5% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

The dosage form contained 0.9 mg ondansetron salt. As seen in FIG. 13, the dosage form released 0.04 mg of ondansetron its 0 to 4 hours, up to 0.01 mg in 0 to 6 hours, up to 0.02 mg in 0 to 8 hours, up to 0.26 mg in 0 to 10 hours, up to 0.56 mg in 0 to 12 hours, up to 0.74 mg in 0 to 14 hours, up to 0.83 mg in 0 to 16 hours, up to 0.88 mg in 0 to 20 hours, and up to 0.88 mg in 0 to 21 hours. The lag time was 8 hours, reached 90% drug release at 15 hours, had a second pulse time (time interval between start to 90% of drug release) of 7 hours, and a slope 0.16 during the drug release from 5% to 70%.

### Example 16

### Trilayer Tablet Release Profile (9 Hour Lag Time—LCT 0409 65-14E SC 19.6%—92:8 CA:Lutrol)

The trilayer core as described in Example 5 was manufactured into a dosage form by being coated with another layer of polymer coating. This second coating layer (semipermeable membrane) includes 92:8 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC-coated trilayer cores to provide a 39.4 mg semipermeable wall (19.6% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

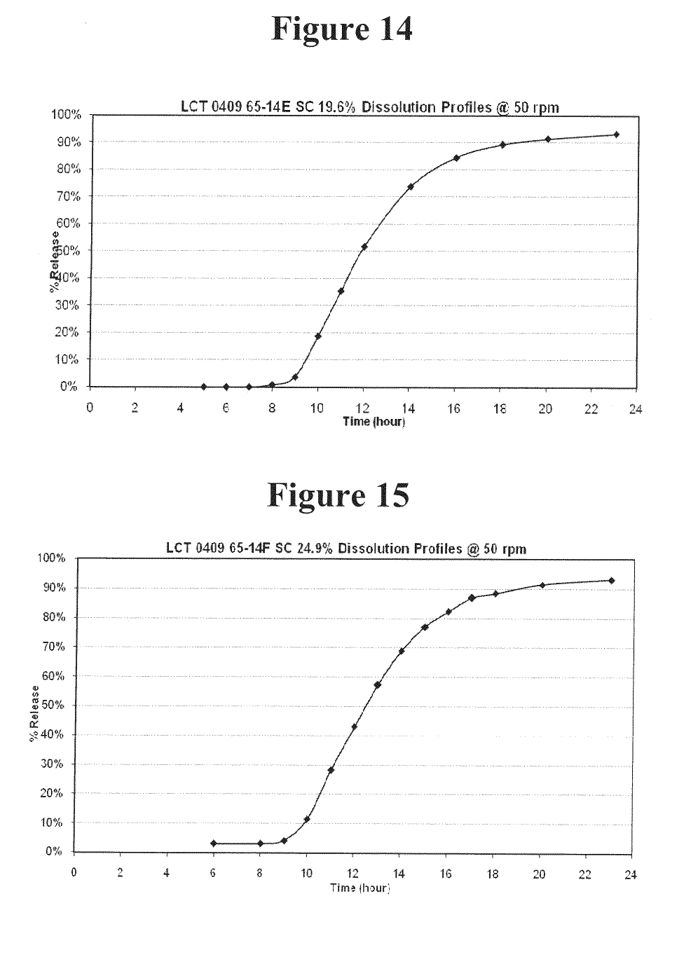

The dosage form contained 0.9 mg ondansetron salt. As seen in FIG. 14, the dosage form released 0.00 mg release in 0 to 6 hours, up to 0.01 mg in 0 to 8 hours, up to 0.17 mg in 0 to 10 hours, up to 0.47 mg in 0 to 12 hours, up to 0.66 mg in 0 to 14 hours, up to 0.76 mg in 0 to 16 hours, up to 0.80 mg in 0 to 18 hours, up to 0.82 in 0 to 20 hours, and up to 0.84 mg in 0 to 23 hours. The lag time was 9 hours, reached 90% drug release at 18 hours, had a second pulse time (time interval between start to 90% of drug release) of 9 hours, and a slope of 0.11 during the drug release from 5% to 70%.

### Example 17

### Trilayer Tablet Release Profile (9 Hour Lag Time—LCT 0409 65-14F SC 24.9%—88:12 CA:Lutrol)

The trilayer core as described in Example 5 was manufactured into a dosage form by being coated with another layer of polymer coating. This second coating layer (semipermeable membrane) includes 88:12 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC-coated trilayer core to provide a 49.8 mg semipermeable wall (24.9%/weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

The dosage form contained 0.9 mg ondansetron salt. As seen in FIG. 15, the dosage form released 0.03 mg of ondansetron in 0 to 6 hours, up to 0.03 mg in 0 to 8 hours, up to 0.10 mg in 0 to 10 hours, up to 0.39 mg in 0 to 12 hours, up to 0.62 mg in 0 to 14 hours, up to 0.74 mg in 0 to 16 hours, up to 0.80 mg in 0 to 18 hours, up to 0.82 in 0 to 2.0 hours, and up to 0.84 mg in 0 to 23 hours. The lag time was 9 hours, reached 90% drug release at 19 hours, had a second pulse time (time interval between start to 90% of drug release) of 10 hours, and a slope of 0.12 during the drug release from 5% to 70%.

### Example 18

### Trilayer Tablet Release Profile (9 Hour Lag Time—LCT 0409 65-14A 18.3%—99:1 CA:PEG)

The trilayer core as described in Example 5 was manufactured into a dosage form by being coated with another layer of polymer coating. This second coating layer (semipermeable membrane) includes 99:1 wt ratio of cellulose acetate and polyethylene glycol 3350, which was dissolved in a cosolvent of acetone and water in 95:5 wt:wt composition to make a 4% solid solution. This composition was sprayed onto and around the HPMC-coated trilayer core to provide a 36.6 mg semipermeable wall (18.3% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

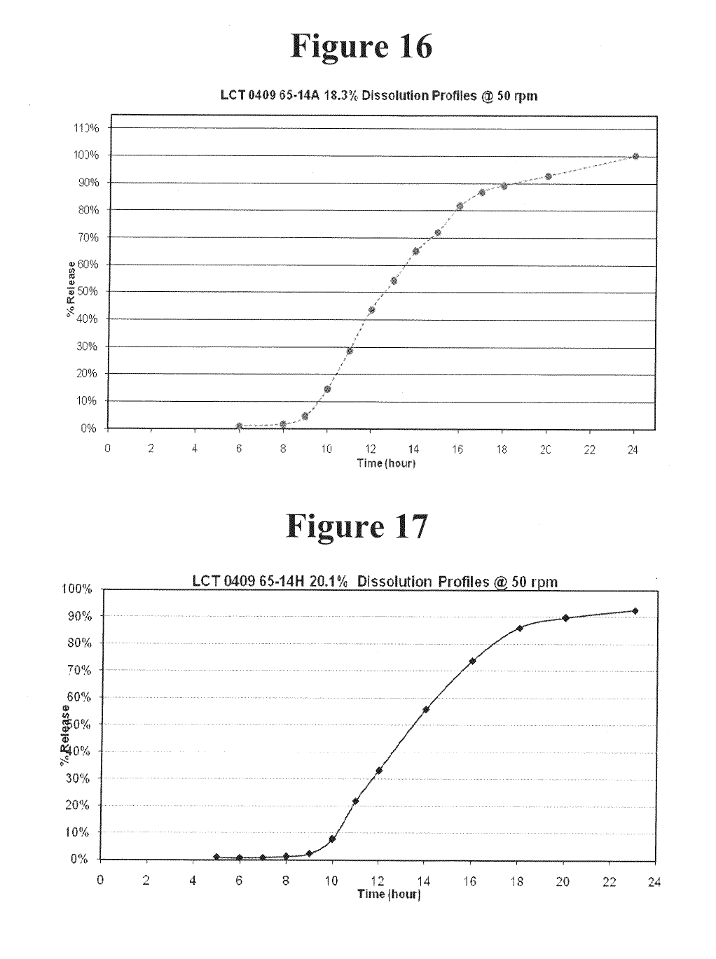

The dosage form contained 0.9 mg ondansetron salt. As seen in FIG. 16, the dosage form released 0.01 mg of ondansetron in 0 to 6 hours, up to 0.02 mg in 0 to 8 hours, up to 0.13 mg in 0 to 10 hours, up to 0.39 mg in 0 to 12 hours, up to 0.59 mg in 0 to 14 hours, up to 0.73 mg in 0 to 16 hours, up to 080 mg in 0 to 18 hours, up to 0.84 mg in 0 to 20 hours, and up to 0.90 mg in 0 to 24 hours. The lag time was 9 hours, reached 90% drug release at 18 hours, had a second pulse time (time interval between start to 90% of drug release) of 9 hours, and a slope of 0.12 during the drug release from 5% to 70%.

### Example 19

### Trilayer Tablet Release Profile (9 Hour Lag Time—LCT 0409 65-14H 20.1%—97:3 CA:PEG)

The trilayer core as described in Example 5 was manufactured into a dosage form by being coated with another layer of polymer coating. This second coating layer (semipermeable membrane) included 97:3 wt ratio of cellulose acetate and polyethylene glycol 3350, which was dissolved in a cosolvent of acetone and water in 95:5 wt:wt composition to make a 4% solid solution. This composition was sprayed onto and around the HPMC coated trilayer cores to provide a 40.1 mg semipermeable wall (20.1% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

The dosage form contained 0.9 mg ondansetron salt. As seen in FIG. 17, the dosage form released 0.01 mg of ondansetron in 0 to 6 hours, up to 0.01 mg in 0 to 8 hours, up to 0.07 mg in 0 to 10 hours, up to 0.30 mg in 0 to 12 hours, up to 0.50 mg in 0 to 14 hours, up to 0.67 mg in 0 to 16 hours, up to 0.78 mg in 0 to 18 hours, up to 0.81 mg in 0 to 20 hours, and up to 0.83 mg in 0 to 24 hours. The lag time was 9 hours, reached 90% drug release at 20 hours, had a second pulse time (time interval between start to 90% of drug release) of 11 hours, and a slope of 0.11 during the drug release from 5% to 70%.

### Example 20

### Trilayer Tablet Release Profile (10 Hour Lag Time—LCT 0409 65-14 SC 20.0%—99:1 CA:PEG)

The trilayer core as described in Example 5 was manufactured into a dosage form by being coated with another layer of polymer coating. This second coating layer (semipermeable membrane) included 99:1 wt ratio of cellulose acetate and polyethylene glycol 3350, which was dissolved in a cosolvent of acetone and water in 95:5 wt:wt composition to make a 4% solid solution. This composition was sprayed onto and around the HPMC-coated trilayer core to provide a 40.0 mg semipermeable wall (20.0% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

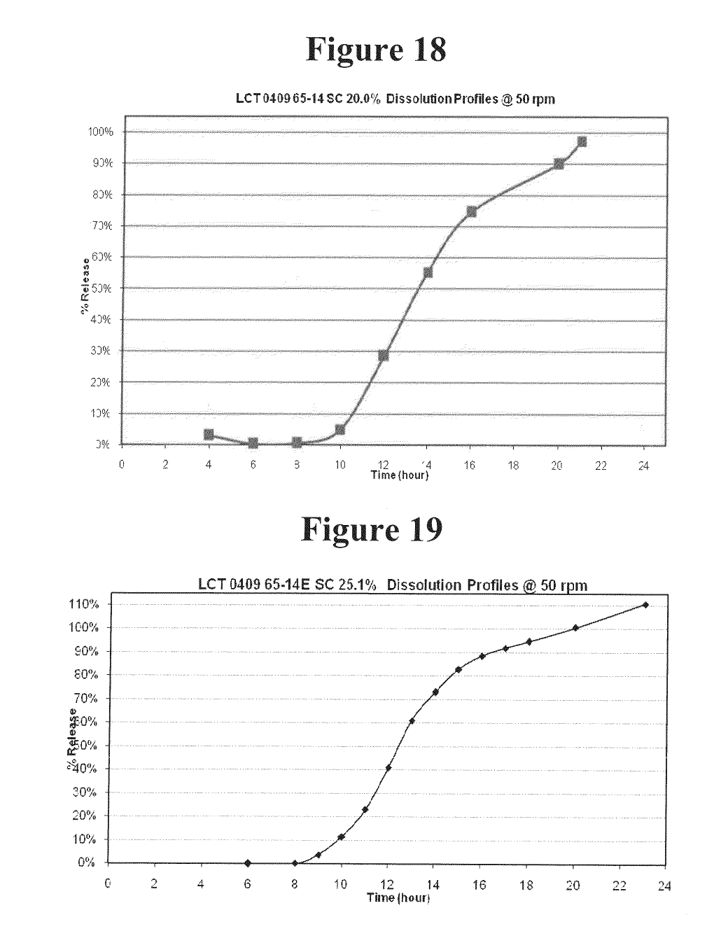

The dosage form contained 0.9 mg ondansetron salt. As seen in FIG. 18, the dosage form released 0.03 mg of ondansetron in 0 to 4 hours, up to 0.00 mg in 0 to 6 hours, up to 0.01 mg in 0 to 8 hours, up to 0.05 mg in 0 to 10 hours, up to 0.26 mg in 0 to 12 hours, up to 0.50 mg in 0 to 14 hours, up to 0.67 mg in 0 to 16 hours, up to 0.81 mg in 0 to 20 hours, and up to 0.88 mg in 0 to 21 hours. The lag time was 10 hours, reached 90% drug release at 2.0 hours, had a second pulse time (time interval between start to 90% of drug release) of 10 hours, and a slope of 0.12 during the drug release from 5% to 70%.

### Example 21

### Trilayer Tablet Release Profile (11 Hour Lag Time—LCT 0409 65-14E SC 25.1%—92:8 CA:Lutrol)

The trilayer core as described in Example 5 was manufactured into a dosage form by being coated with another layer of polymer coating. This second coating layer (semipermeable membrane) included 92:8 wt ratio of cellulose acetate and copolymer of polyethylene oxide and polypropylene oxide (Lutrol), which was dissolved in a cosolvent of acetone and water in 95:5 wt:wt composition to make a 4% solid solution. This composition was sprayed onto and around the HPMC-coated trilayer core to provide a 50.1 mg semipermeable wall (25.1% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

The dosage form contained 0.9 mg ondansetron salt. As seen in FIG. 19, the dosage form released 0.00 mg of ondansetron in 0 to 6 hours, up to 0.00 mg in 0 to 8 hours, up to 0.10 mg in 0 to 10 hours, up to 0.37 mg in 0 to 12 hours, up to 0.66 mg in 0 to 14 hours, up to 0.80 mg in 0 to 16 hours, up to 0.85 mg in 0 to 18 hours, up to 0.91 in 0 to 20 hours, and up to 1.00 mg in 0 to 23 hours. The lag time was 8 hours, reached 90% drug release at 17 hours, had a second pulse time (time interval between start to 90% of drug release) of 6 hours, and a slope of 0.19 during the drug release from 5% to 70%.

### Example 22

### Trilayer Tablet Release Profile (11 Hour Lag Time—LCT 0409 65-14H 26.6%—97:3 CA:PEG)

The trilayer core as described in Example 5 was manufactured into a dosage fatal by being coated with another layer of polymer coating. This second coating layer (semipermeable membrane) included 97:3 wt ratio of cellulose acetate and polyethylene glycol 3350, which was dissolved in a cosolvent of acetone and water in 95:5 wt:wt composition to make a 4% solid solution. This composition was sprayed onto and around the HPMC-coated trilayer core to provide a 53.4 mg semipermeable wall (26.6% weight gain of the core). The semipermeable-walled trilayer tablet was drilled to provide a 20 mil (0.51 mm) orifice to contact the first (placebo) layer and the exterior of the dosage form. The residual solvent was removed by drying for 72 hours at 45° C. and 45% relative humidity. The dosage form was then dried further for 6 hours at 45° C. to remove excess moisture.

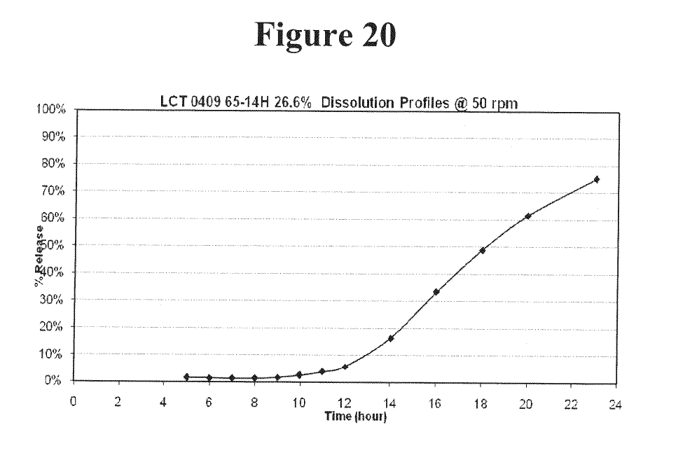

The dosage form contained 0.9 mg ondansetron salt. As seen in FIG. 20, the dosage form released 0.01 mg of ondansetron in 0 to 6 hours, up to 0.01 mg in 0 to 8 hours, up to 0.02 mg in 0 to 10 hours, up to 0.05 rang in 0 to 12 hours, up to 0.15 mg in 0 to 14 hours, up to 0.30 mg in 0 to 16 hours, up to 0.44 mg in 0 to 18 hours, up to 0.56 mg in 0 to 20 hours, and up to 0.68 mg in 0 to 23 hours. The lag time was 8 hours, reached 90% drug release at 26 hours, had a second pulse time (time interval between start to 90% of drug release) of 15 hours, and a slope of 0.07 during the drug release from 5% to 70%.

### Example 23

### Trilayer Tablets with and without Subcoats

Cracking of the semipermeable membrane coating outside of the tablet at a point along the edge of the trilayer tablet was often observed after 5-6 hours of drug release testing. A study was performed to assess the use of a subcoat—a layer of polymer coating between the semipermeable membrane and the trilayer tablet—on cracking of the semipermeable membrane or swelling of the tablet during drug release.

### Blending and Compression of the Trilayer Core

The first layer: The first (placebo) layer contains polyethylene oxide, croscarmellose sodium, silicon dioxide (Sylloid 244FP), and magnesium stearate. All ingredients were screened separately through a 30 mesh screen and then dry blended excluding magnesium stearate in a conventional blender for 13 minutes followed by the addition of magnesium stearate with the blending continued for another 5 minutes to yield a homogenous blend.

The second layer: The drug layer contains ondansetron hydrochloride, polyethylene oxide, povidone, croscarmellose sodium, silicon dioxide (Sylloid 244FP), sodium chloride, magnesium stearate, butylated hydroxytoluene, and ferric oxide. All ingredients were screened separately through a 30 mesh screen and then dry blended excluding magnesium stearate in a conventional blender for 13 minutes followed by the addition of magnesium stearate with the blending continued for another 5 minutes to yield a homogenous blend.

The third layer: The push layer contains 700K polyethylene oxide, povidone, silicon dioxide (Sylloid 244FP), sodium chloride, magnesium stearate, butylated hydroxytoluene, and ferric oxide. All ingredients were screened separately through a 30 mesh screen then dry blended excluding magnesium stearate in a conventional blender for 13 minutes followed by the addition of magnesium stearate with the blending continued for another 5 minutes to yield a homogenous blend.

Compression of the trilayer core: The first, second, and third layers were weighed out according to the amount listed in Table 2 thr each formulation. In the order listed, the blends were added into a punch-die set and tamped in between each layer. The three layers were then compressed under a pressure head of 500 psi using a Carver press.

TABLE 2
Compositions of the Trilayer Tablets With and Without Subcoat

| | without subcoat | | | with subcoat | |
|---|---|---|---|---|---|
| | MW | MW | MW | MW | MW |
| Components | 8.8% | 12.1% | 18.8% | 10.8% | 15% |
| First Layer | | | | | |
| Polyethylene oxide 300K | 46.75 | 46.75 | 46.75 | 46.75 | 46.75 |
| Croscarmellose Sodium | 2 | 2 | 2 | 2 | 2 |
| Silicon Dioxide (Sylloid 244FP) | 1 | 1 | 1 | 1 | 1 |
| Magnesium Stearate | 0.25 | 0.25 | 0.25 | 0.25 | 0.25 |
| Total First layer weight | 50 | 50 | 50 | 50 | 50 |
| Second Layer | | | | | |
| Ondansetron HCl | 0.9 | 0.9 | 0.9 | 0.9 | 0.9 |
| Polyethylene oxide 100K | — | — | — | 31.83 | 31.83 |
| Polyethylene oxide 200K | 31.82 | 31.82 | 31.82 | — | — |
| Povidone (K29-32) | 2.5 | 2.5 | 2.5 | 2.51 | 2.51 |
| Croscarmellose Sodium | 3 | 3 | 3 | 3 | 3 |
| Silicon Dioxide (Sylloid 244FP) | 1 | 1 | 1 | 1.01 | 1.01 |
| Sodium Chloride | 10 | 10 | 10 | 10.01 | 10.01 |
| Magnesium Stearate | 0.25 | 0.25 | 0.25 | 0.26 | 0.26 |
| Butylated Hydroxytoluene | 0.03 | 0.03 | 0.03 | 0.03 | 0.03 |
| Ferric oxide | 0.5 | 0.5 | 0.5 | 0.5 | 0.5 |
| Total second layer weight | 50 | 50 | 50 | 50 | 50 |
| Push Layer | | | | | |
| Polyethylene oxide 7000K | 72.45 | 72.45 | 72.45 | 72.45 | 72.45 |
| Povidone (K29-32) | 5 | 5 | 5 | 5 | 5 |
| Silicon Dioxide (Sylloid 244FP) | 1.33 | 1.33 | 1.33 | 1.33 | 1.33 |
| Sodium Chloride | 20 | 20 | 20 | 20 | 20 |
| Magnesium Stearate | 0.5 | 0.5 | 0.5 | 0.5 | 0.5 |
| Butylated Hydroxytoluene | 0.05 | 0.05 | 0.05 | 0.05 | 0.05 |
| Ferric oxide | 0.67 | 0.67 | 0.67 | 0.67 | 0.67 |
| Total Push Layer weight | 100 | 100 | 100 | 100 | 100 |
| Subcoat | | | | | |
| Hydroxypropylmethyl cellulose | — | — | — | 9.2 | 9.2 |
| Total Subcoat layer weight | — | — | — | 9.2 | 9.2 |
| Semipermeable membrane | | | | | |
| Cellulose acetate (398-10) | 17.4 | 23.8 | 37.2 | 21.4 | 29.7 |
| Polyethylene glycol 3350 | 0.2 | 0.4 | 0.4 | 0.2 | 0.3 |
| Total semipermeable membrane weight | 17.6 | 24.2 | 37.6 | 21.6 | 30 |
| Total Tablet weight | 217.6 | 224.2 | 237.6 | 230.8 | 239.2 |

Subcoat coating procedure: The trilayer tablet was coated with a polymer coating. This subcoat was prepared from a 10% aqueous solution of hydroxypropylmethylcellulose (HPMC). The solution was sprayed onto the trilayer cores as prepared above to provide a 10 mg subcoat layer (5% weight gain of the core). The coated tablet with HPMC was dried for 1 hour at 48° C.

Semipermeable Membrane Coating and Exit Orifice procedure: The semipermeable membrane comprises cellulose acetate and polyethylene glycol 3350 which was dissolved in a cosolvent comprising of acetone and water. The coating was done at LDCS pan coater of Vector Corporation. The wall-forming composition was sprayed onto and around the sub-coated trilayer cores. Next, the semipermeable walled, trilayer tablet was to provide an osmotically controlled extrusion pathway for the tablet contents. The residual solvent from the coating was removed by drying.

Dissolution method: Tablets were dropped into 500 mL of de-aerated de-ionized water (DI H₂O), 37° C. using USP dissolution apparatus 2 (paddles) at 50 rpm. Samples were collected and transferred to HPLC vials at 6, 8, 9, 10, 11, 12, 13, 14, 15, 16, 17, 18, 20, and 24 hours.

HPLC method: HPLC analysis was performed on a Waters 2695 HPLC system (SN B00SD6 021M/G07SHC442A) equipped with a Waters 2996 PDA detector (SN E06296 830M) or Waters 2487 dual wavelength absorbance detector (SN G07487681M). A. Zorbax SB-CN, 3 μm, 4.5×50 mm column was used (SN USJF001656). The mobile phase consists of 45% Methanol and 55% of a 25 mM H₂PO₄ pH=6.3 buffer. Samples were analyzed at 250 nm, 100 μl injections, with a flow rate of 1.0 mL/min.

### Results

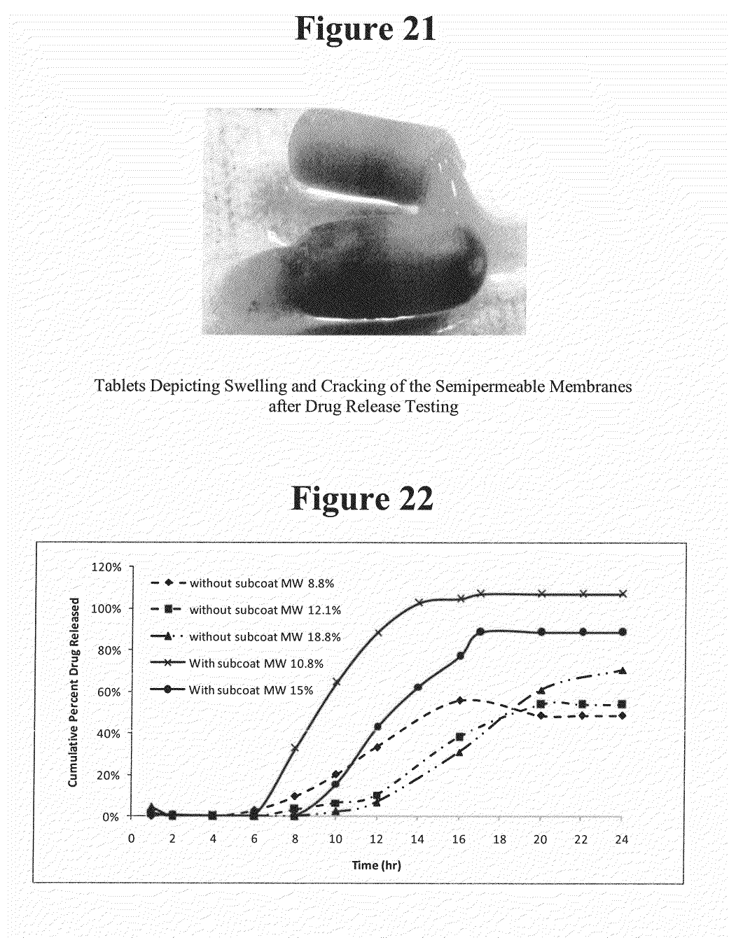

FIG. 21 depicts the trilayer tablets without the subcoat layer after the drug release testing. As shown, the semipermeable membrane, on the outside, was found to crack along the edge of the tablet during testing. Also the tablets were found to be swollen compared to the original tablets. Both swelling of tablets and cracking of the semipermeable membrane affected the drug release profiles adversely, as shown in FIG. 22. A high amount of residual drug (30-50%) was leftover in the tablet at the end of 24 hours. The profile shape depicted 2 distinct slopes. The lag time was shorter than anticipated. Using a subcoat layer between the semipermeable membrane and the trilayer tablet core alleviated both the swelling of the tablet and cracking of the semipermeable membrane. The release profiles from trilayer tablets made with a formulation (composition listed in Table 2) resulted in longer lag times, as desired and zero order release of the drug from the second layer. The residual drug leftover was less than 10%, as shown in release profiles in FIG. 22.

All publications, patent applications, and patents cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A pharmaceutical composition comprising a core, a semi-permeable membrane disposed generally around the core, an orifice in the semi-permeable membrane in fluid communication with the core, and a coating comprising a first therapeutically effective dose of ondansetron or a pharmaceutically acceptable salt disposed generally around the semi-permeable membrane,
wherein the core comprises first, second, and third layers, wherein the first layer is in fluid communication with the orifice, wherein the second layer comprises a second therapeutically effective dose of ondansetron or a pharmaceutically acceptable salt and is located adjacent to the first layer, and wherein the third layer is located adjacent the second layer, and
wherein the first layer has a higher viscosity than the second layer.

2-16. (canceled)

17. A method for treating OCD, comprising the steps of:
providing a dosage form comprising a core, a semi-permeable membrane disposed generally around the core, an orifice in the semi-permeable membrane in fluid communication with the core, and a coating comprising a first therapeutically effective dose of ondansetron or a pharmaceutically acceptable salt disposed generally around the semi-permeable membrane; and
administering the dosage form to a patient suffering from OCD,
wherein the core comprises first, second, and third layers, wherein the first layer is in fluid communication with the orifice, wherein the second layer comprises a second therapeutically effective dose of ondansetron or a pharmaceutically acceptable salt and is located adjacent to the first layer, and wherein the third layer is located adjacent the second layer, and
wherein the first layer has a higher viscosity than the second layer.

18. The method of claim 17, wherein the first layer has a viscosity between about 50 and about 20,000 cps.

19. The method of claim 17, wherein the first layer has a viscosity between about 50 and about 1500 cps.

20. The method of claim 17, wherein the first layer has a viscosity between about 600 and about 1200 cps.

21. The method of claim 17, wherein the first layer has a viscosity between about 500 and about 1000 cps.

22. The method of claim 17, wherein the second layer has a viscosity between about 30 and about 10,000 cps.

23. The method of claim 17, wherein the second layer has a viscosity between about 30 and about 1,000 cps.

24. The method of claim 17, wherein the second layer has a viscosity between about 30 and about 100 cps.

25. The method of claim 17, wherein the second layer has a viscosity between about 5 and about 50 cps.

26. The method of claim 17, wherein the first layer comprises polyethylene oxide with an average molecular weight of about 300K.

27. The method of claim 17, wherein the first layer comprises polyethylene oxide with an average molecular weight between about 200K to about 1000K.

28. The method of claim 17, wherein the second layer comprises polyethylene oxide with an average molecular weight of about 100K.

29. The method of claim 17, wherein the second layer comprises polyethylene oxide with an average molecular weight between about 100K to about 600K.

30. The method of claim 17, wherein the first and second therapeutically effective doses are each about 0.125 to about 1 mg.

31. The method of claim 17, wherein the first therapeutically effective dose of ondansetron is released from the dosage form within 2 hours of administration of the dosage form.

32. The method of claim 17, wherein the second therapeutically effective dose of ondansetron is released from the dosage form not less than 8 hours after administration of the dosage form.

33. The method of claim 17, wherein a first dose-normalized Cₘₐₓ corresponding to the release of the first therapeutically effective dose is between about 0.5 ng/ml and about 6 ng/ml.

34. The method of claim 17, wherein a second dose-normalized Cₘₐₓ corresponding to the release of the second therapeutically effective dose is between about 0.5 ng/ml and about 6 ng/ml.

35. The method of claim 17, wherein a first Tₘₐₓ corresponding to the release of the first therapeutically effective dose occurs between about 0.1 hours and about 6 hours of administration.

36. The method of claim 17, wherein a second Tₘₐₓ corresponding to the release of the second therapeutically effective dose occurs between about 6 hours and about 20 hours of administration.

37. The method of claim 17, wherein a first dose-normalized corresponding to a trough level after the release of the first therapeutically effective dose is between about 0.1 ng/ml and about 1 ng/ml.

38. The method of claim 17, wherein a second dose-normalized Cₘᵢₙ corresponding to a trough level after the release of the second therapeutically effective dose is between about 0.1 ng/ml and about 1 ng/ml.

39. The method of claim 17, wherein a first Tₘᵢₙ corresponding to a trough level after the release of the first therapeutically effective dose occurs between about 4 hours and about 10 hours of administration.

40. The method of claim 17, wherein the first and third layers are substantially free of ondansetron.

41. The method of claim 17, wherein there is no substantial accumulation of ondansetron after 24-hours.

42. A pharmaceutical composition comprising a core, a semi-permeable membrane disposed generally around the core, an orifice in the semi-permeable membrane in fluid communication with the core, and a coating comprising a first therapeutically effective dose of ondansetron or a pharmaceutically acceptable salt disposed generally around the semi-permeable membrane,
wherein the core comprises first, second, and third layers, wherein the first layer is in fluid communication with the orifice, wherein the second layer comprises a second therapeutically effective dose of ondansetron or a pharmaceutically acceptable salt and is located adjacent to the first layer, and wherein the third layer is located adjacent the second layer,
wherein each of the first and second layers comprise polyethylene oxide having an average molecular weight and wherein the average molecular weight of the polyethylene oxide in the first layer is higher than the average molecular weight of the polyethylene oxide in the second layer.

43-50. (canceled)

51. A method for treating OCD, comprising the steps of:
providing a dosage form comprising a core, a semi-permeable membrane disposed generally around the core, an orifice in the semi-permeable membrane in fluid communication with the core, and a coating comprising a first therapeutically effective dose of ondansetron or a pharmaceutically acceptable salt disposed generally around the semi-permeable membrane; and
administering the dosage form to a patient suffering from OCD.
wherein the core comprises first, second, and third layers, wherein the first layer is in fluid communication with the orifice, wherein the second layer comprises a second therapeutically effective dose of ondansetron or a pharmaceutically acceptable salt and is located adjacent to the first layer, and
wherein the third layer is located adjacent the second layer, wherein each of the first and second layers comprise polyethylene oxide having an average molecular weight and wherein the average molecular weight of the polyethylene oxide in the first layer is higher than the average molecular weight of the polyethylene oxide in the second layer.

52-68. (canceled)

69. A method for treating OCD, comprising the steps of:
providing a dosage form comprising a first compartment and a second compartment, the first compartment comprising ondansetron in a dose of about 0.125 mg to about 1.0 mg, the second compartment comprising ondansetron in a dose of about 0.125 mg to about 1.0 mg; and
administering the dosage form to a patient suffering from OCD,
wherein the first compartment is formulated for release within 2 hours, and
wherein the second compartment comprising ondansetron is formulated for release in not less than 8 hours, and
wherein there is no substantial accumulation of ondansetron after 24-hours following administration.

70-81. (canceled)

82. A pharmaceutical composition comprising a core, a semi-permeable membrane disposed generally around the core, an orifice in the semi-permeable membrane in fluid communication with the core, and a coating comprising a first therapeutically effective dose of ondansetron or a pharmaceutically acceptable salt disposed generally around the semi-permeable membrane,
wherein the core comprises first, second, and third layers, wherein the first layer is in fluid communication with the orifice, wherein the second layer comprises a second therapeutically effective dose of ondansetron or a pharmaceutically acceptable salt and is located adjacent to the first layer, and wherein the third layer is located adjacent the second layer.

83-94. (canceled)

95. A method for treating OCD, comprising the steps of providing a dosage form comprising a first compartment and a second compartment, each of the first and second compartments comprising a therapeutically effective dose of ondansetron; and
administering the dosage form to a patient suffering from OCD,
wherein the first and second compartments initiate release at different times and result in a first dose-normalized Cₘₐₓ, a first Tₘₐₓ, a second dose-normalized Cₘₐₓ, and a second Tₘₐₓ, wherein the first and second dose-normalized Cₘₐₓ's are each between about 0.5 ng/ml and about 6 ng/ml, the first Tₘₐₓ is between about 0.1 hours and about 6 hours of administration, and the second Tₘₐₓ is between about 6 hours and about 20 hours of administration.

96-103. (canceled)
